(19) United States
(12) Patent Application Publication
(10) Pub. No.: US 2012/0083531 A1
(43) Pub. Date: Apr. 5, 2012

(54) CALCIUM CITRATE AND CALCIUM LACTATE FORMULATIONS FOR ALTERATION OF BIOPHYSICAL PROPERTIES OF MUCOSAL LINING

(76) Inventors: Robert W. Clarke, Medfield, MA (US); Richard Batycky, Newton, MA (US); David L. Hava, Natick, MA (US); Michal M. Lipp, Framingham, MA (US)

(21) Appl. No.: 13/259,666

(22) PCT Filed: Mar. 26, 2010
(86) PCT No.: PCT/US10/28914

§ 371 Date: Nov. 21, 2011

Related U.S. Application Data
(60) Provisional application No. 61/163,772, filed on Mar. 26, 2009.
(60) Provisional application No. 61/267,747, filed on Dec. 8, 2009.

(51) Int. Cl. A61K 31/194; A61P 11/00; A61K 31/19; A61P 31/00; C07C 59/08; C07C 59/265

(52) U.S. Cl. 514/574; 562/589; 562/584; 514/557

(57) ABSTRACT

The present invention relates to pharmaceutical compositions suitable for inhalation, comprising as an active ingredient calcium lactate or calcium citrate. The invention also relates to methods of treating, preventing, and reducing the spread of an infection of the respiratory tract, comprising administering a pharmaceutical composition that comprises calcium lactate or calcium citrate as an active ingredient.

### RELATED APPLICATIONS

This application claims the benefit of U.S. Provisional Application No. 61/163,772, filed on Mar. 26, 2009 and U.S. Provisional Application No. 61/267,747, filed on Dec. 8, 2009. The entire teachings of the above applications are incorporated herein by reference.

### BACKGROUND OF THE INVENTION

Millions of people worldwide suffer from diseases or conditions that could be treated or prevented by altering the mucosal lining. Many organs have a liquid mucosal lining whose biophysical properties can facilitate or impede normal function. A wide array of adverse health effects have been associated with the properties of a mucosal lining, for instance, particles “shed” from the upper airway mucosal lining fluid (UAL) during normal exhalation may carry viable, infectious bacterial or viral pathogens, such as Severe Acute Respiratory Syndrome (SARS) coronavirus, influenza, and tuberculosis, which are capable of spreading to healthy individuals through inhalation; the surface tension of the UAL has been shown to play a role in obstructive sleep apnea syndrome; and alteration of the mucosal lining of the intestinal tract by viruses/mycobacteria may lead to inflammatory bowel disease over time. Controlled alteration of the mucosal lining's biophysical properties can effectively treat and/or prevent many of these adverse health effects.

Previously, non-surfactant solutions that alter physical properties of lung mucus lining fluid have been shown to treat and prevent the transmission of some diseases and conditions. Primarily aerosolized formulations containing isotonic saline solution or hypertonic saline solution have been used to limit bioaerosol formation and/or spread of infection. Dry powders provide substantial advantages over liquid formulations (e.g., ease of delivery, etc.). However, many of these formulations have issues that make them undesirable as dry powders, such as challenges related to processing the salt into a dry powder respirable form, the low solubility of many salts, the high hygroscopicity of the more soluble salts, and exothermic qualities that limit inhalation treatment.

Thus, a need exists to develop additional formulations that are capable of altering the mucosal lining, without the undesirable properties listed above. It is therefore necessary to pursue additional formulation work (i.e., combining calcium chloride with other excipients to reduce the load in the final product) and to identify other salts to achieve a desired calcium, sodium and chlorine ionic content in the formulations. Ideally, these new formulations would include large amounts of calcium salts in a stable dry powder that could be aerosolized for pulmonary delivery.

### SUMMARY OF THE INVENTION

The invention relates to a pharmaceutical composition comprising as an active ingredient, a calcium salt selected from the group consisting of calcium lactate and calcium citrate, wherein the pharmaceutical composition is suitable for inhalation.

In some embodiments, the pharmaceutical composition further comprises a sodium salt. The sodium salt can be sodium chloride, sodium acetate, sodium bicarbonate, sodium carbonate, sodium sulfate, sodium stearate, sodium ascorbate, sodium benzoate, sodium biphosphate, sodium phosphate, sodium bisulfite, sodium citrate, sodium lactate, sodium borate, sodium gluconate or sodium metasilicate. In a preferred embodiment, the sodium salt is sodium chloride.

In some embodiments the pharmaceutical composition comprises calcium and sodium in a ratio of 8:1 (mole:mole). In other embodiments, the pharmaceutical composition comprises calcium and sodium in a ratio of 1:2 (mole:mole). In other embodiments, the pharmaceutical composition comprises calcium and sodium in a ratio of 1:1.3 (mole:mole). In some embodiments, the pharmaceutical composition is formulated to deliver a calcium dose of about 0.01 mg/kg body weight/dose to about 10 mg/kg body weight/dose to the lungs. In other embodiments, the pharmaceutical composition is formulated to provide a sodium dose of about 0.001 mg/kg body weight/dose to about 10 mg/kg body weight/dose to the lungs. In some embodiments, the pharmaceutical composition is formulated to deliver a calcium dose of about 0.01 mg/kg body weight/dose to about 10 mg/kg body weight/dose to the nasal cavity. In other embodiments, the pharmaceutical composition is formulated to provide a sodium dose of about 0.001 mg/kg body weight/dose to about 10 mg/kg body weight/dose to the nasal cavity.

In some embodiments the composition is a liquid formulation. The liquid formulation can be a solution or a suspension. In some embodiments the calcium lactate is present from about 0.1% to about 20% (w/v).

In other embodiments the pharmaceutical composition is a dry powder. The calcium salt can be calcium lactate or calcium citrate. In some embodiments the calcium salt is present from about 0.5% to about 90% (w/w).

The pharmaceutical composition can further comprise an additional therapeutic agent. The pharmaceutical composition can be a unit dose composition.

The invention also relates to a method for treating an infection of the respiratory tract, comprising administering to an individual having an infection of the respiratory tract or exhibiting symptoms of a respiratory tract infection, an effective amount of a pharmaceutical composition that is suitable for inhalation comprising calcium lactate or calcium citrate as an active ingredient.

The invention further relates to a method for prophylaxis of an infection of the respiratory tract, comprising administering to an individual at risk of contracting an infection of the respiratory tract an effective amount of a pharmaceutical composition that is suitable for inhalation comprising calcium lactate or calcium citrate as an active ingredient.

The invention also relates to a method for reducing the spread of an infection of the respiratory tract, comprising administering to an individual having an infection of the respiratory tract, exhibiting symptoms of a respiratory tract infection or at risk of contracting a respiratory tract infection an effective amount of a pharmaceutical composition that is suitable for inhalation comprising calcium lactate or calcium citrate as an active ingredient.

In some embodiments, the respiratory tract infection is an infection caused by a bacteria selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Stenotrophomonas maltophilia, Burkholderia spp. and combinations thereof. In other embodiments, the infection is an infection caused by a virus selected from the group consisting of influenza virus, respiratory syncytial virus, adenovirus, metapneumovirus, cytomegalovirus, parainfluenza virus, rhinovirus, herpes simplex virus, SARS-coronavirus and smallpox.

The invention also relates to a method for treatment of chronic pulmonary disease including asthma (e.g., allergic/atopic, childhood, late-onset, cough-variant, or chronic obstructive), airway hyperresponsiveness, allergic rhinitis (seasonal or non-seasonal), bronchiectasis, chronic bronchitis, emphysema, chronic obstructive pulmonary disease, cystic fibrosis, early life wheezing, and the like. The salt formulations are effective for blocking acute exacerbation of a chronic pulmonary disease in an individual. Exemplary pulmonary diseases include asthma (e.g., allergic/atopic, childhood, late-onset, cough-variant, or chronic obstructive), airway hyperresponsiveness, allergic rhinitis (seasonal or non-seasonal), bronchiectasis, chronic bronchitis, emphysema, chronic obstructive pulmonary disease, cystic fibrosis, early life wheezing, and the like. The invention further relates to a method for blocking acute exacerbations of chronic pulmonary disease and preventing bronchoconstriction and bronchospasms due to antigen exposure (e.g., allergen, pathogen, and other environmental stimulants).

### BRIEF DESCRIPTION OF THE DRAWINGS

FIG. 1 is a graph showing that cells exposed to the calcium citrate dry powder had reduced influenza titers 24 hours after dosing compared to both untreated control cells (air) and cells exposed to control leucine dry powder.

FIG. 2 is a graph showing that liquid formulations of calcium lactate inhibit influenza infection. The calcium lactate formulations significantly reduced viral infection compared to the untreated control (air).

FIG. 3 is a graph showing that cells treated with a calcium lactate dry powder reduced influenza infection as shown by reduced viral titer 24 hours after dosing compared to the untreated control (air).

FIG. 4 is a schematic of the pass-through model.

FIG. 5A is a graph showing the results of the bacterial pass-through model with exposure to dry powders. Calcium sulfate (4.5 μg Ca/cm² delivered dose) reduced bacterial movement through sodium alginate mimetic.

FIG. 5B is a graph showing the results of the bacterial pass-through model with exposure to dry powders. The calcium salt formulations tested contained 0 μg, 4.3 μg, 6.4 μg or 10 μg of calcium. Calcium sulfate (4.3 μg Ca/cm² delivered dose), calcium acetate (10 μg Ca/cm² delivered dose) and calcium lactate (6.4 μg Ca/cm² delivered dose) salts reduce bacterial movement through sodium alginate mimetic.

FIG. 6 is a graph showing that calcium lactate reduces influenza infection in a dose responsive manner. Each of the calcium lactate dry powder concentrations reduced the viral titer compared to the air control.

FIG. 7 is a graph showing that liquid formulation of calcium lactate at a concentration of 0.116M modestly reduces bacterial burden in a pneumonia infection.

FIGS. 8A-C are graphs showing antiviral activity of calcium dry powder formulations against different viral pathogens. Calu-3 cells exposed to no formulation were used as a control and compared to Calu-3 cells exposed to PUR111, PUR112, and PUR113. The concentration of virus released by cells exposed to each aerosol formulation was quantified. Bars represent the mean and standard deviation of duplicate wells for each test.

### DETAILED DESCRIPTION OF THE INVENTION

The invention relates to calcium lactate and calcium citrate formulations. The formulations can also include sodium salts. Specifically, formulations may be comprised of calcium citrate and/or calcium lactate, with or without a sodium salt (e.g., sodium chloride). The formulations may be liquid (e.g., solution, suspension) or dry powder. The formulations are processed and formulated such that their physical and aerodynamic properties are appropriate for delivery to the respiratory tract (e.g., upper respiratory tract, respiratory airways, lungs). As described herein, the results of studies into the inhibition, prevention and prevention of spread of infections of the respiratory tract are shown.

The term “respiratory tract infection” is a term of art that refers to upper respiratory infections (e.g., infections of the nasal cavity, pharynx, larynx) and lower respiratory infections (e.g., infections of the trachea, primary bronchi, lungs) and combinations thereof. Typical symptoms associated with respiratory tract infections include nasal congestion, cough, running nose, sore throat, fever, facial pressure, sneezing, chest pain and difficulty breathing.

The term “pneumonia” is a term of art that refers to an inflammatory illness of the lung. Pneumonia can result from a variety of causes, including infection with bacteria, viruses, fungi, or parasites, and chemical or physical injury to the lungs. Typical symptoms associated with pneumonia include cough, chest pain, fever and difficulty breathing. Clinical diagnosis of pneumonia is well-known in the art and may include x-ray and/or examination of sputum.

The term “bacterial pneumonia” refers to pneumonia caused by bacterial infection, including for example, infection of the respiratory tract by Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Stenotrophomonas maltophilia, Burkholderia spp. and combinations thereof.

The term “viral pneumonia” refers to pneumonia caused by a viral infection. Viruses that commonly cause viral pneumonia include, for example, influenza virus, respiratory syncytial virus (RSV), adenovirus, and metapneumovirus. Herpes simplex virus is a rare cause of pneumonia for the general population, but is more common in newborns. People with weakened immune systems are also at risk for pneumonia caused by cytomegalovirus (CMV).

The term “aerosol” as used herein refers to any preparation of a fine mist of particles (including liquid and non-liquid particles, e.g., dry powders), typically with a volume median geometric diameter of about 0.1 to about 30 microns or a mass median aerodynamic diameter of between about 0.5 and about 10 microns. Preferably the volume median geometric diameter for the aerosol particles is less than about 10 microns. The preferred volume median geometric diameter for aerosol particles is about 5 microns. For example, the aerosol can contain particles that have a volume median geometric diameter between about 0.1 and about 30 microns, between about 0.5 and about 20 microns, between about 0.5 and about 10 microns, between about 1.0 and about 3.0 microns, between about 1.0 and 5.0 microns, between about 1.0 and 10.0 microns, between about 5.0 and 15.0 microns. Preferably the mass median aerodynamic diameter is between about 0.5 and about 10 microns, between about 1.0 and about 3.0 microns, or between about 1.0 and 5.0 microns.

The term “respiratory tract” as used herein includes the upper respiratory tract (e.g., nasal passages, nasal cavity, throat, pharynx), respiratory airways (e.g., larynx, trachea, bronchi, bronchioles) and lungs (e.g., respiratory bronchioles, alveolar ducts, alveolar sacs, alveoli).

As used herein, “1×” tonicity refers to a solution that is isotonic relative to normal human blood and cells. Solutions that are hypotonic or hypertonic in comparison to normal human blood and cells are described relative to a 1× solution using an appropriate multiplier. For example, a hypotonic solution may have 0.1×, 0.25× or 0.5× tonicity, and a hypertonic solution may have 2×, 3×, 4×, 5×, 6×, 7×, 8×, 9× or 10× tonicity.

The term “dry powder” as used herein refers to a composition that contains finely dispersed respirable dry particles that are capable of being dispersed in an inhalation device and subsequently inhaled by a subject. Such dry powder or dry particle may contain up to about 15% water or other solvent, or be substantially free of water or other solvent, or be anhydrous.

### Formulations

The invention relates to salt formulations that comprise calcium lactate and/or calcium citrate as an active ingredient, and can optionally contain additional salts or agents. The salt formulations are for administration to the respiratory tract, for example as an aerosol. The salt formulations are effective for treatment, prophylaxis and/or reducing contagion of infectious diseases of the respiratory tract (e.g., viral infections, bacterial infections). The salt formulations are effective for treatment of chronic pulmonary diseases. Exemplary pulmonary diseases include asthma (e.g., allergic/atopic, childhood, late-onset, cough-variant, or chronic obstructive), airway hyperresponsiveness, allergic rhinitis (seasonal or non-seasonal), bronchiectasis, chronic bronchitis, emphysema, chronic obstructive pulmonary disease, cystic fibrosis, early life wheezing, and the like. The salt formulations are effective for blocking acute exacerbation of chronic pulmonary disease in an individual. The salt formulations are effective for preventing bronchoconstriction and bronchospasms due to antigen (e.g., allergen, pathogen, and other environmental stimulants).

The salt formulations are effective for altering the biophysical properties of the mucosal lining of the respiratory tract. These properties include, for example, gelation at the mucus surface, surface tension of the mucosal lining, surface elasticity and/or viscosity of the mucosal lining, bulk elasticity and/or viscosity of the mucosal lining. Without wishing to be bound by a particular theory, it is believed that the benefits produced by the salt formulations and the methods described herein (e.g., therapeutic and prophylactic benefits), result from an increase in the amount of calcium cation (Ca²⁺ provided by the calcium salts in the salt formulation) in the respiratory tract (e.g., lung mucus or airway lining fluid) after administration of the salt formulation. The salt formulations slow the passage of antigens through the airway lining fluid, thereby reducing the exposure to antigen that may trigger inflammation and subsequent bronchoconstriction and bronchospams resulting from an immune response.

Calcium citrate and calcium lactate have several advantages over alternative calcium salts for use in formulations for the pulmonary administration of calcium in both liquid and dry powder forms. In particular, calcium citrate and calcium lactate possess sufficient aqueous solubility to allow for their processing into respirable dry powders via spray-drying and to facilitate their dissolution upon deposition in the lungs, yet possess a low enough hygroscopicity to allow for the production of dry powders with high calcium salt loads that are relatively physically stable upon exposure to normal and elevated humidities. Additionally, calcium chloride (e.g., calcium chloride dehydrate) has a large exothermic heat of solution, and substantial heat is produced when it dissolves. This creates a risk of burns if calcium chloride is administered to mucus membranes. Calcium citrate and calcium lactate also do not possess the limitations associated with calcium chloride salts with respect to the exothermic heat of soultion. Finally, citrate and lactate ions are considered to be safe and acceptable for inclusion in pharmaceutical compositions.

Further, with respect to dry powder formulations, calcium citrate and calcium lactate possess a preferred combination of properties over those of other calcium salt forms, including calcium chloride salt forms. Calcium citrate and calcium lactate are (i) capable of being processed into a dry powder form of a particle size distribution suitable for pulmonary administration, (ii) possess sufficient physicochemical stability in dry powder form to facilitate the production of a dry powder that is dispersible and physically stable over a range of conditions, including upon exposure to elevated humidity conditions, (iii) capable of undergoing rapid dissolution upon deposition in the lungs and (iv) do not possess properties that can result in poor tolerability or adverse events, such as possessing a significant exothermic heat of mixing, etc.

In addition to calcium lactate or calcium citrate, the salt formulations can include any non-toxic salt form of the elements sodium, potassium, magnesium, calcium, aluminum, silicon, scandium, titanium, vanadium, chromium, cobalt, nickel, copper, manganese, zinc, tin, silver and similar elements. The salt formulation can be in any desired form, such as a solution, emulsion, suspension, or a dry powder. Preferred salt formulations, such as solutions and dry powders, can be aerosolized. Preferred salt formulations contain a sodium salt (e.g., saline (0.15 M NaCl or 0.9% solution)), in addition to calcium lactate or calcium citrate. When the formulation comprises calcium lactate and a sodium salt, or calcium citrate and a sodium salt, it can, if desired, also contain one or more other salts. The salt formulations can comprise multiple doses or be a unit dose composition as desired.

Suitable sodium salts include, for example, sodium chloride, sodium acetate, sodium bicarbonate, sodium carbonate, sodium sulfate, sodium stearate, sodium ascorbate, sodium benzoate, sodium biphosphate, sodium phosphate, sodium bisulfite, sodium citrate, sodium lactate, sodium borate, sodium gluconate, sodium metasilicate, and the like, or combinations thereof.

Suitable calcium salts include, for example, calcium chloride, calcium carbonate, calcium acetate, calcium phosphate, calcium alginate, calcium stearate, calcium sorbate, calcium sulfate, calcium gluconate, calcium citrate, calcium lactate, and the like, or combinations thereof.

Suitable magnesium salts include, for example, magnesium carbonate, magnesium acetate, magnesium phosphate, magnesium alginate, magnesium sorbate, magnesium sulfate, magnesium gluconate, magnesium stearate, magnesium trisilicate, magnesium chloride, magnesium citrate, magnesium lactate and the like, or combinations thereof.

Suitable potassium salts include, for example, potassium bicarbonate, potassium chloride, potassium citrate, potassium borate, potassium bisulfite, potassium biphosphate, potassium alginate, potassium benzoate, and the like, or combinations thereof. Additional suitable salts include cupric sulfate, chromium chloride, stannous chloride, and similar salts.

Other suitable salts include zinc chloride, aluminum chloride and silver chloride.

The salt formulation is generally prepared in or comprises a physiologically acceptable carrier or excipient. For salt formulations in the form of solutions, suspensions or emulsions, any suitable carrier or excipient can be included. Suitable carriers include, for example, aqueous, alcoholic/aqueous, and alcohol solutions, emulsions or suspensions, including water, saline, ethanol/water solution, ethanol solution, buffered media, propellants and the like. For salt formulations in the form of dry powders, suitable carriers or excipients include, for example, sugars (e.g., lactose, trehalose), sugar alcohols (e.g., mannitol, xylitol, sorbitol), amino acids (e.g., glycine, alanine, leucine, isoleucine), dipalmitoylphosphosphatidylcholine (DPPC), diphosphatidyl glycerol (DPPG), 1,2-Dipalmitoyl-sn-glycero-3-phospho-L-serine (DPPS), 1,2-Dipalmitoyl-sn-glycero-3-phosphocholine (DSPC), 1,2-Distearoyl-sn-glycero-3-phosphoethanolamine (DSPE), 1-palmitoyl-2-oleoylphosphatidylcholine (POPC), fatty alcohols, polyoxyethylene-9-lauryl ether, surface active fatty, acids, sorbitan trioleate (Span 85), glycocholate, surfactin, poloxomers, sorbitan fatty acid esters, tyloxapol, phospholipids, alkylated sugars, sodium phosphate, maltodextrin, human serum albumin (e.g., recombinant human serum albumin), biodegradable polymers (e.g., PLGA), dextran, dextrin, and the like. If desired, the salt formulations can also contain additives, preservatives, or fluid, nutrient or electrolyte replenishers (See, generally, Remington's Pharmaceutical Sciences, 17th Edition, Mack Publishing Co., PA, 1985).

The salt formulations preferably contain a concentration of calcium lactate or calcium citrate that permits convenient administration of an effective amount of the formulation to the respiratory tract. For example, it is generally desirable that liquid formulations not be so dilute so as to require a large amount of the formulation to be nebulized in order to deliver an effective amount to the respiratory tract of a subject. Long administration periods are disfavored, and generally the formulation should be concentrated enough to permit an effective amount to be administered to the respiratory tract (e.g., by inhalation of aerosolized formulation, such as nebulized liquid or aerosolized dry powder) or nasal cavity in no more than about 120 minutes, no more than about 90 minutes, no more than about 60 minutes, no more than about 45 minutes, no more than about 30 minutes, no more than about 25 minutes, no more than about 20 minutes, no more than about 15 minutes, no more than about 10 minutes, no more than about 7.5 minutes, no more than about 5 minutes, no more than about 4 minutes, no more than about 3 minutes, no more than about 2 minutes, no more than about 1 minute, no more than about 45 seconds, or no more than about 30 seconds. For example, a liquid calcium lactate or calcium citrate formulation can contain about 0.01% to about 30% calcium lactate or calcium citrate (w/v), between 0.1% to about 20% calcium lactate or calcium citrate (w/v), or between 0.1% to about 10% calcium lactate or calcium citrate (w/v). Liquid formulations can contain about 0.001M to about 1.5M calcium lactate or calcium citrate, about 0.01M to about 1.0M calcium lactate or calcium citrate, about 0.01M to about 0.9M calcium lactate or calcium citrate, about 0.01M to about 0.8M calcium lactate or calcium citrate, about 0.01M to about 0.7M calcium lactate or calcium citrate, about 0.01M to about 0.6M calcium lactate or calcium citrate, about 0.01M to about 0.5M calcium lactate or calcium citrate, about 0.01M to about 0.4M calcium lactate or calcium citrate, about 0.01M to about 0.3M calcium lactate or calcium citrate, about 0.01M to about 0.2M calcium lactate or calcium citrate, about 0.1M to about 1.0M calcium lactate or calcium citrate, about 0.1M to about 0.9M calcium lactate or calcium citrate, about 0.1M to about 0.8M calcium lactate or calcium citrate, about 0.1M to about 0.7M calcium lactate or calcium citrate, about 0.1M to about 0.6M calcium lactate or calcium citrate, about 0.1M to about 0.5M calcium lactate or calcium citrate, about 0.1M to about 0.4M calcium lactate or calcium citrate, about 0.1M to about 0.3M calcium lactate or calcium citrate, or about 0.1M to about 0.2M calcium lactate or calcium citrate. The solubility of calcium citrate and calcium lactate can limit the preparation of solutions. In such situations, the liquid formulation can be in the form of a suspension that contains the equivalent amount of calcium salt that would be needed to achieve the desired molar concentration.

Dry powder formulations can contain at least about 5% calcium lactate or calcium citrate by weight, at least about 10% calcium lactate or calcium citrate by weight, at least about 15% calcium salt lactate or calcium citrate weight, at least about 19.5% calcium lactate or calcium citrate by weight, at least about 20% calcium lactate or calcium citrate by weight, at least about 22% calcium lactate or calcium citrate by weight, at least about 25.5% calcium lactate or calcium citrate by weight, at least about 30% calcium lactate or calcium citrate by weight, at least about 35% calcium lactate or calcium citrate by weight, at least about 40% calcium lactate or calcium citrate by weight, at least about 45% calcium lactate or calcium citrate by weight, at least about 50% calcium lactate or calcium citrate by weight, at least about 55% calcium lactate or calcium citrate by weight, at least about 60% calcium lactate or calcium citrate by weight, at least about 65% calcium lactate or calcium citrate by weight, at least about 70% calcium lactate or calcium citrate by weight, at least about 75% calcium lactate or calcium citrate by weight, at least about 80% calcium lactate or calcium citrate by weight, at least about 85% calcium lactate or calcium citrate by weight, at least about 90% calcium lactate or calcium citrate by weight, at least about 95% calcium lactate or calcium citrate by weight, at least about 96% calcium lactate or calcium citrate by weight, at least about 97% calcium lactate or calcium citrate by weight, at least about 98% calcium lactate or calcium citrate by weight, or at least about 99% calcium lactate or calcium citrate by weight. For example, some dry powder formulations contain about 20% to about 80% calcium lactate or calcium citrate by weight, about 20% to about 70% calcium lactate or calcium citrate by weight, about 20% to about 60% calcium lactate or calcium citrate by weight, or can consist substantially of calcium lactate or calcium citrate.

Alternatively or in addition, such dry powder formulations may contain a calcium salt which provides Ca⁺² in an amount of at least about 5% Ca⁺² by weight, at least about 7% Ca⁺² by weight, at least about 10% Ca⁺² by weight, at least about 11% Ca⁺² by weight, at least about 12% Ca⁺² by weight, at least about 13% Ca⁺² by weight, at least about 14% Ca⁺² by weight, at least about 15% Ca⁺² by weight, at least about 17% Ca⁺² by weight, at least about 20% Ca⁺² by weight, at least about 25% Ca⁺² by weight, at least about 30% Ca⁺² by weight, at least about 35% Ca⁺² by weight, at least about 40% Ca⁺² by weight, at least about 45% Ca⁺² by weight, at least about 50% Ca⁺² by weight, at least about 55% Ca⁺² by weight, at least about 60% Ca⁺² by weight, at least about 65% Ca⁺² by weight or at least about 70% Ca⁺² by weight.

When a dry powder salt formulation contains calcium lactate or calcium citrate and a sodium salt the amount of sodium salt in the dry powder formulation can be dependent upon the desired calcium:sodium ratio. For example, the dry powder formulation may contain at least about 1.6% sodium salt by weight, at least about 5% sodium salt by weight, at least about 10% sodium salt by weight, at least about 13% sodium salt by weight, at least about 15% sodium salt by weight, at least about 20% sodium salt by weight, at least about 24.4% sodium salt by weight, at least about 28% sodium salt by weight, at least about 30% sodium salt by weight, at least about 30.5% sodium salt by weight, at least about 35% sodium salt by weight, at least about 40% sodium salt by weight, at least about 45% sodium salt by weight, at least about 50% sodium salt by weight, at least about 55% sodium salt by weight, or at least about 60% sodium salt by weight.

Alternatively or in addition, dry powder salt formulations may contain a sodium salt which provides Na⁺ in an amount of at least about 0.1% Na⁺ by weight, at least about 0.5% Na⁺ by weight, at least about 1% Na⁺ by weight, at least about 2% Na⁺ by weight, at least about 3% Na⁺ by weight, at least about 4% Na⁺ by weight, at least about 5% Na⁺ by weight, at least about 6% Na⁺ by weight, at least about 7% Na⁺ by weight, at least about 8% Na⁺ by weight, at least about 9% Na⁺ by weight, at least about 10% Na⁺ by weight, at least about 11% Na⁺ by weight, at least about 12% Na⁺ by weight, at least about 14% Na⁺ by weight, at least about 16% Na⁺ by weight, at least about 18% Na⁺ by weight, at least about 20% Na⁺ by weight, at least about 22% Na⁺ by weight, at least about 25% Na⁺ by weight, at least about 27% Na⁺ by weight, at least about 29% Na⁺ by weight, at least about 32% Na⁺ by weight, at least about 35% Na⁺ by weight, at least about 40% Na⁺ by weight, at least about 45% Na⁺ by weight, at least about 50% Na⁺ by weight, or at least about 55% Na⁺ by weight.

Certain calcium salts provide two or more moles of Ca²⁺ per mole of calcium salt upon dissolution. Such calcium salts may be particularly suitable to produce liquid or dry powder formulations that are dense in calcium, and therefore, can deliver an effective amount of cation (e.g., Ca²⁺, Na⁺, or Ca²⁺ and Na⁺). For example, one mole of calcium citrate provides three moles of Ca²⁺ upon dissolution. It is also generally preferred that the calcium salt is a salt with a low molecular weight and/or contain low molecular weight anions. Low molecular weight calcium salts, such as calcium salts that contain calcium ions and low molecular weight anions, are calcium dense relative to high molecular salts and calcium salts that contain high molecular weight anions. It is generally preferred that the calcium salt has a molecular weight of less than about 1000 g/mol, less than about 950 g/mol, less than about 900 g/mol, less than about 850 g/mol, less than about 800 g/mol, less than about 750 g/mol, less than about 700 g/mol, less than about 650 g/mol, less than about 600 g/mol, less than about 550 g/mol, less than about 510 g/mol, less than about 500 g/mol, less than about 450 g/mol, less than about 400 g/mol, less than about 350 g/mol, less than about 300 g/mol, less than about 250 g/mol, less than about 200 g/mol, less than about 150 g/mol, less than about 125 g/mol, or less than about 100 g/mol. In addition or alternatively, it is generally preferred that the calcium ion contributes a substantial portion of the weight to the overall weight of the calcium salt. It is generally preferred that the calcium ion weigh at least 10% of the overall calcium salt, at least 16%, at least 20%, at least 24.5%, at least 26%, at least 31%, at least 35%, or at least 38% of the overall calcium salt.

Some salt formulations contain a calcium salt in which the weight ratio of calcium to the overall weight of said calcium salt is between about 0.1 to about 0.5. For example, the weight ratio of calcium to the overall weight of said calcium salt is between about 0.15 to about 0.5, between about 0.18 to about 0.5, between about 0.2 to about 0.5, between about 0.25 to about 0.5, between about 0.27 to about 0.5, between about 0.3 to about 0.5, between about 0.35 to about 0.5, between about 0.37 to about 0.5, or between about 0.4 to about 0.5.

Some salt formulations contain calcium lactate and a sodium salt, for example 0.12 M calcium lactate or calcium citrate in 0.15 M sodium chloride, or 3.7% (w/v) calcium lactate in 0.90% saline. Some salt formulations that contain calcium lactate or calcium citrate and a sodium salt are characterized by the ratio of calcium:sodium (mole:mole). Suitable ratios of calcium:sodium (mole:mole) can range from about 0.1:1 to about 32:1, about 0.5:1 to about 16:1, about 2:1 to about 16:1, about 4:1 to about 12:1, about 1:1 to about 8:1. For example, the ratio of calcium:sodium (mole:mole) can be about 0.77:1, about 1:1, about 1:1.3, about 1:2, about 2:1, about 4:1, about 8:1 or about 16:1 (mole:mole). In particular examples, the salt formulations contain calcium lactate or calcium citrate and sodium chloride, and have a calcium:sodium ratio of about 8:1 (mole:mole). Aqueous liquid salt formulations of this type can vary in tonicity and in the concentrations of calcium salt and sodium salt that are present in the formulation For example, the salt formulation can contain calcium lactate and sodium chloride at tonicities and molarities listed in Table 1

TABLE 1
Liquid calcium lactate and sodium chloride formulations

| Ca-lactate + | | Calcium lactate | | Sodium chloride | | Calcium ion | | Sodium ion | |
|---|---|---|---|---|---|---|---|---|---|
| NaCl (8:1 Ca:Na | | Conc | Molarity | Conc | Molarity | Conc | Molarity | Conc | Molarity |
| molar ratio) | | Ca-lact (wt %) | Ca-lact (M) | NaCl (wt %) | NaCl (M) | Ca (wt %) | Ca (M) | Na (wt %) | Na (M) |
| Tonicity | 1X | 3.85 | 0.18 | 0.13 | 0.022 | 0.71 | 0.18 | 0.051 | 0.022 |
| | 2X | 7.71 | 0.35 | 0.26 | 0.044 | 1.42 | 0.35 | 0.10 | 0.044 |
| | 4X | 15.4 | 0.71 | 0.52 | 0.088 | 2.83 | 0.71 | 0.20 | 0.088 |
| | 8X | 30.8 | 1.41 | 1.0 | 0.18 | 5.66 | 1.41 | 0.41 | 0.18 |

In certain aspects, the salt formulation that contains a calcium salt and a sodium salt and the ratio of Ca⁺² to Na⁺ is from about 4:1 (mole:mole) to about 16:1 (mole:mole). For example, the formulations can contain a ratio of Ca⁺² to Na⁺ from about 5:1 (mole:mole) to about 16:1 (mole:mole), from about 6:1 (mole:mole) to about 16:1 (mole:mole), from about 7:1 (mole:mole) to about 16:1 (mole:mole), from about 8:1 (mole:mole) to about 16:1 (mole:mole), from about 9:1 (mole:mole) to about 16:1 (mole:mole), from about 10:1 (mole:mole) to about 16:1 (mole:mole), from about 11:1 (mole:mole) to about 16:1 (mole:mole), from about 12:1 (mole:mole) to about 16:1 (mole:mole), from about 13:1 (mole:mole) to about 16:1 (mole:mole), from about 14:1 (mole:mole) to about 16:1 (mole:mole), from about 15:1 (mole:mole) to about 16:1 (mole:mole).

In certain aspects, the salt formulation that contains a calcium salt and a sodium salt and the ratio of Ca⁺² to Na⁺ is from about 4:1 (mole:mole) to about 5:1 (mole:mole), from about 4:1 (mole:mole) to about 6:1 (mole:mole), from about 4:1 (mole:mole) to about 7:1 (mole:mole), from about 4:1 (mole:mole) to about 8:1 (mole:mole), from about 4:1 (mole:mole) to about 9:1 (mole:mole), from about 4:1 (mole:mole) to about 10:1 (mole:mole), from about 4:1 (mole:mole) to about 11:1 (mole:mole), from about 4:1 (mole:mole) to about 12:1 (mole:mole), from about 4:1 (mole:mole) to about 13:1 (mole:mole), from about 4:1 (mole:mole) to about 14:1 (mole:mole), from about 4:1 (mole:mole) to about 15:1 (mole:mole).

The salt formulations can contain a ratio of Ca⁺² to Na⁺ from about 4:1 (mole:mole) to about 12:1 (mole:mole), from about 5:1 (mole:mole) to about 11:1 (mole:mole), from about 6:1 (mole:mole) to about 10:1 (mole:mole), from about 7:1 (mole:mole) to about 9:1 (mole:mole).

In particular examples, the ratio of Ca⁺² to Na⁺ is about 4:1 (mole:mole), about 4.5:1 (mole:mole), about 5:1 (mole:mole), about 5.5:1 (mole:mole), about 6:1 (mole:mole), about 6.5:1 (mole:mole), 7:1 (mole:mole), about 7.5:1 (mole:mole), about 8:1 (mole:mole), about 8.5:1 (mole:mole), about 9:1 (mole:mole), about 9.5:1 (mole:mole), about 10:1 (mole:mole), about 10.5:1 (mole:mole), about 11:1 (mole:mole), about 11.5:1 (mole:mole), about 12:1 (mole:mole), about 12.5:1 (mole:mole), about 13:1 (mole:mole), about 13.5:1 (mole:mole), about 14:1 (mole:mole), about 14.5:1 (mole:mole), about 15:1 (mole:mole), about 15.5:1 (mole:mole), or about 16:1 (mole:mole).

In more particular examples, the ratio of Ca⁺² to Na⁺ is about 8:1 (mole:mole) or about 16:1 (mole:mole).

The salt formulation can be hypotonic, isotonic or hypertonic as desired. For example, any of the salt formulations described herein may have about 0.1× tonicity, about 0.25× tonicity, about 0.5× tonicity, about 1× tonicity, about 2× tonicity, about 3× tonicity, about 4× tonicity, about 5× tonicity, about 6× tonicity, about 7× tonicity, about 8× tonicity, about 9× tonicity, about 10× tonicity, at least about 1× tonicity, at least about 2× tonicity, at least about 3× tonicity, at least about 4× tonicity, at least about 5× tonicity, at least about 6× tonicity, at least about 7× tonicity, at least about 8× tonicity, at least about 9× tonicity, at least about 10× tonicity, between about 0.1× to about 1×, between about 0.1× to about 0.5×, between about 0.5× to about 2×, between about 1× to about 4×, between about 1× to about 2×, between about 2× to about 10×, or between about 4× to about 8×.

If desired, the salt formulation can include one or more additional agents, such as mucoactive or mucolytic agents, surfactants, antibiotics, antivirals, antihistamines, cough suppressants, bronchodilators, anti-inflammatory agents, steroids, vaccines, adjuvants, expectorants, macromolecules, therapeutics that are helpful for chronic maintenance of CF.

Examples of suitable mucoactive or mucolytic agents include MUC5AC and MUC5B mucins, DNA-ase, N-acetylcysteine (NAC), cysteine, nacystelyn, dornase alfa, gelsolin, heparin, heparin sulfate, P2Y2 agonists (e.g. UTP, INS365), hypertonic saline, and mannitol.

Suitable surfactants include L-alpha-phosphatidylcholine dipalmitoyl (“DPPC”), diphosphatidyl glycerol (DPPG), 1,2-Dipalmitoyl-sn-glycero-3-phospho-L-serine (DPPS), 1,2-Dipalmitoyl-sn-glycero-3-phosphocholine (DSPC), 1,2-Distearoyl-sn-glycero-3-phosphoethanolamine (DSPE), 1-palmitoyl-2-oleoylphosphatidylcholine (POPC), fatty alcohols, polyoxyethylene-9-lauryl ether, surface active fatty, acids, sorbitan trioleate (Span 85), glycocholate, surfactin, poloxomers, sorbitan fatty acid esters, tyloxapol, phospholipids, and alkylated sugars.

If desired, the salt formulation can contain an antibiotic. For example, salt formulations for treating bacterial pneumonia or VAT, can further comprise an antibiotic, such as a macrolide (e.g., azithromycin, clarithromycin and erythromycin), a tetracycline (e.g., doxycycline, tigecycline), a fluoroquinolone (e.g., gemifloxacin, levofloxacin, ciprofloxacin and mocifloxacin), a cephalosporin (e.g., ceftriaxone, defotaxime, ceftazidime, cefepime), a penicillin (e.g., amoxicillin, amoxicillin with clavulanate, ampicillin, piperacillin, and ticarcillin) optionally with a β-lactamase inhibitor (e.g., sulbactam, tazobactam and clavulanic acid), such as ampicillin-sulbactam, piperacillin-tazobactam and ticarcillin with clavulanate, an aminoglycoside (e.g., amikacin, arbekacin, gentamicin, kanamycin, neomycin, netilmicin, paromomycin, rhodostreptomycin, streptomycin, tobramycin, and apramycin), a penem or carbapenem (e.g. doripenem, ertapenem, imipenem and meropenem), a monobactam (e.g., aztreonam), an oxazolidinone (e.g., linezolid), vancomycin, glycopeptide antibiotics (e.g. telavancin), tuberculosis-mycobacterium antibiotics and the like.

If desired, the salt formulation can contain an agent for treating infections with mycobacteria, such as Mycobacterium tuberculosis. Suitable agents for treating infections with mycobacteria (e.g., M. tuberculosis) include an aminoglycoside (e.g. capreomycin, kanamycin, streptomycin), a fluoroquinolone (e.g. ciprofloxacin, levofloxacin, moxifloxacin), isozianid and isozianid analogs (e.g. ethionamide), aminosalicylate, cycloserine, diarylquinoline, ethambutol, pyrazinamide, protionamide, rifampin, and the like.

If desired, the salt formulation can contain a suitable antiviral agent, such as oseltamivir, zanamavir amantidine or rimantadine, ribavirin, gancyclovir, valgancyclovir, foscavir, Cytogam® (Cytomegalovirus Immune Globulin), pleconaril, rupintrivir, palivizumab, motavizumab, cytarabine, docosanol, denotivir, cidofovir, and acyclovir. The salt formulation can contain a suitable anti-influenza agent, such as zanamivir, oseltamivir, amantadine, or rimantadine.

Suitable antihistamines include clemastine, asalastine, loratadine, fexofenadine and the like.

Suitable cough suppressants include benzonatate, benproperine, clobutinal, diphenhydramine, dextromethorphan, dibunate, fedrilate, glaucine, oxalamine, piperidione, opiods such as codine and the like.

Suitable brochodilators include short-acting beta₂ agonists, long-acting beta₂ agonists (LABA), long-acting muscarinic anagonists (LAMA), combinations of LABAs and LAMAs, methylxanthines, and the like. Suitable short-active beta₂ agonists include albuterol, epinephrine, pirbuterol, levalbuterol, metaproteronol, maxair, and the like. Suitable LABAs include salmeterol, formoterol and isomers (e.g. arformoterol), clenbuterol, tulobuterol, vilanterol (Revolair™), indacaterol, and the like. Examples of LAMAs include tiotroprium, glycopyrrolate, aclidinium, ipratropium and the like. Examples of combinations of LABAs and LAMAs include indacaterol with glycopyrrolate, indacaterol with tiotropium, and the like. Examples of methylxanthine include theophylline, and the like.

Suitable anti-inflammatory agents include leukotriene inhibitors, PDE4 inhibitors, other anti-inflammatory agents, and the like. Suitable leukotriene inhibitors include montelukast (cystinyl leukotriene inhibitors), masilukast, zafirleukast (leukotriene D4 and E4 receptor inhibitors), zileuton (5-lipoxygenase inhibitors), and the like. Suitable PDE4 inhibitors include cilomilast, roflumilast, and the like. Other anti-inflammatory agents include omalizumab (anti IgE immunoglobulin), IL-13 and IL-13 receptor inhibitors (such as AMG-317, MILR1444A, CAT-354, QAX576, IMA-638, Anrukinzumab, IMA-026, MK-6105, DOM-0910 and the like), IL-4 and IL-4 receptor inhibitors (such as Pitrakinra, AER-003, AIR-645, APG-201, DOM-0919 and the like), IL-1 inhibitors such as canakinumab, CRTh2 receptor antagonists such as AZD1981 (from AstraZeneca), neutrophil elastase inhibitor such as AZD9668 (from AstraZeneca), P38 kinase inhibitor such as losmapimed, and the like.

Suitable steroids include corticosteroids, combinations of corticosteroids and LABAs, combinations of corticosteroids and LAMAs, and the like. Suitable corticosteroids include budesonide, fluticasone, flunisolide, triamcinolone, beclomethasone, mometasone, ciclesonide, dexamethasone, and the like. Combinations of corticosteroids and LABAs include salmeterol with fluticasone, formoterol with budesonide, formoterol with fluticasone, formoterol with mometasone, indacaterol with mometasone, and the like.

Suitable expectorants include guaifenesin, guaiacolculfonate, ammonium chloride, potassium iodide, tyloxapol, antimony pentasulfide and the like.

Suitable vaccines include nasally inhaled influenza vaccines and the like.

Suitable macromolecules include proteins and large peptides, polysaccharides and oligosaccharides, and DNA and RNA nucleic acid molecules and their analogs having therapeutic, prophylactic or diagnostic activities. Proteins can include antibodies such as monoclonal antibody. Nucleic acid molecules include genes, antisense molecules such as siRNAs that bind to complementary DNA, RNA, or ribosomes to inhibit transcription or translation.

Selected therapeutics that are helpful for chronic maintenance of CF include antibiotics/macrolide antibiotics, bronchodilators, inhaled LABAs, and agents to promote airway secretion clearance. Suitable examples of antibiotics/macrolide antibiotics include tobramycin, azithromycin, ciprofloxacin, colistin, and the like. Suitable examples of bronchodilators include inhaled short-acting beta₂ agonists such as albuterol, and the like. Suitable examples of inhaled LABAs include salmeterol, formoterol, and the like. Suitable examples of agents to promote airway secretion clearance include dornase alfa, hypertonic saline, and the like.

Dry powder formulations are prepared with the appropriate particle diameter, surface roughness, and density for localized delivery to selected regions of the respiratory tract. For example, higher density or larger particles may be used for upper airway delivery. Similarly, a mixture of different sized particles can be administered to target different regions of the lung in one administration.

As used herein, the phrase “aerodynamically light particles” refers to particles having a tap density less than about 0.4 g/cm³. The tap density of particles of a dry powder may be obtained by the standard USP tap density measurement. Tap density is a common measure of the envelope mass density. The envelope mass density of an isotropic particle is defined as the mass of the particle divided by the minimum sphere envelope volume in which it can be enclosed. Features contributing to low tap density include irregular surface texture and porous structure.

Dry powder formulations (“DPFs”) with large particle size have improved flowability characteristics, such as less aggregation (Visser, J., Powder Technology 58: 1-10 (1989)), easier aerosolization, and potentially less phagocytosis. Rudt, S, and R. H. Muller. J. Controlled Release, 22: 263-272 (1992); Tabata Y., and Y. Ikada. J. Biomed. Mater. Res. 22: 837-858 (1988). However, if deposition in the pulmonary tract beyond the oral cavity is desired, it is generally understood that the DPFs should have a mass median aerodynamic diameter of less than 10 microns, and more preferably less than 5 microns. Therefore dry powder aerosols for inhalation therapy are generally produced with volume mean geometric diameters primarily in the range of less than 10 microns, and preferably less than 5 microns, although dry powders that have any desired range in aerodynamic diameter can be produced. Ganderton D., J. Biopharmaceutical Sciences, 3:101-105 (1992); Gonda, I. “Plysico-Chemical Principles in Aerosol Delivery.” in Topics in Pharmaceutical Sciences 1991, Crommelin, D. J. and K. K. Midha, Eds., Medpharm Scientific Publishers, Stuttgart, pp. 95-115 (1992). Large “carrier” particles (containing no salt formulation) can be co-delivered with therapeutic aerosols to aid in achieving efficient aerosolization among other possible benefits. French, D. L., Edwards, D. A. and Niven, R. W., J. Aerosol Sci. 27: 769-783 (1996). Particles with degradation and release times ranging from seconds to months can be designed and fabricated by established methods in the art.

Generally, salt formulations that are dry powders may be produced by spray drying, freeze drying, jet milling, single and double emulsion solvent evaporation, and super-critical fluids. Preferably, salt formulations are produced by spray drying, which entails preparing a liquid feed stock containing the salt and other components of the formulation, spraying the liquid feed stock into a closed chamber, and removing the solvent with a heated gas steam. Generally, milling is not preferred for the production of respirable dry powders due to poor control over the particle size distribution.

Spray dried powders that contain salts with sufficient solubility in water or aqueous solvents, such as calcium chloride and calcium lactate, can be readily prepared using conventional methods. Some salts, such as calcium citrate and calcium carbonate, have relatively low solubility in water and other aqueous solvents. Spray dried powders that contain such salts can be prepared using any suitable method. One suitable method involves combining other more soluble salts in solution and permitting reaction (precipitation reaction) to produce the desired salt for the dry powder formulation. For example, if a dry powder formulation comprising calcium citrate and sodium chloride is desired, a solution containing the high solubility salts calcium chloride and sodium citrate can be prepared. The precipitation reaction leading to calcium citrate is 3 CaCl₂+2Na₃Cit→Ca₃Cit₂+6NaCl. It is preferable that the sodium salt is fully dissolved before the calcium salt is added and that the solution is continuously stirred. The precipitation reaction can be allowed to go to completion or stopped before completion, e.g., by spray drying the solution, as desired.

Alternatively, two saturated or sub-saturated solutions are fed into a static mixer in order to obtain a saturated or supersaturated solution post-static mixing. Preferably, the post-spray drying solution is supersaturated. The two solutions may be aqueous or organic, but are preferably substantially aqueous. The post-static mixing solution is then fed into the atomizing unit of a spray dryer. In a preferable embodiment, the post-static mixing solution is immediately fed into the atomizer unit. Some examples of an atomizer unit include a two-fluid nozzle, a rotary atomizer, or a pressure nozzle. Preferably, the atomizer unit is a two-fluid nozzle. In one embodiment, the two-fluid nozzle is an internally mixing nozzle, meaning that the gas impinges on the liquid feed before exiting to the most outward orifice. In another embodiment, the two-fluid nozzle is an externally mixing nozzle, meaning that the gas impinges on the liquid feed after exiting the most outward orifice.

The resulting solution may appear clear with fully dissolved salts or a precipitate may form. Depending on reaction conditions, a precipitate may form quickly or over time. Solutions that contain a light precipitate that results in formation of a stable homogenous suspension can be spray dried.

Dry powder formulations can also be prepared by blending individual components into the final formulation. For example, a first dry powder that contains a calcium salt can be blended with a second dry powder that contains a sodium salt to produce a dry powder salt formulation that contains a calcium salt and a sodium salt. If desired, additional dry powders that contain excipients (e.g., lactose) and/or other active ingredients (e.g., antibiotic, antiviral) can be included in the blend. The blend can contain any desired relative amounts or ratios of salts, excipients and other ingredients (e.g., antibiotics, antivirals).

If desired, dry powders can be prepared using polymers that are tailored to optimize particle characteristics including: i) interactions between the agent (e.g., salt) to be delivered and the polymer to provide stabilization of the agent and retention of activity upon delivery; ii) rate of polymer degradation and thus agent release profile; iii) surface characteristics and targeting capabilities via chemical modification; and iv) particle porosity. Polymeric particles may be prepared using single and double emulsion solvent evaporation, spray drying, solvent extraction, solvent evaporation, phase separation, simple and complex coacervation, interfacial polymerization, jet milling and other methods well known to those of ordinary skill in the art. Particles may be made using methods for making microspheres or microcapsules known in the art.

Dry powder salt formulations that contain a calcium salt generally contain at least about 3% calcium salt by weight, at least about 5% calcium salt by weight, 10% calcium salt by weight, about 15% calcium salt by weight, at least about 19.5% calcium salt by weight, at least about 20% calcium salt by weight, at least about 22% calcium salt by weight, at least about 25.5% calcium salt by weight, at least about 30% calcium salt by weight, at least about 37% calcium salt by weight, at least about 40% calcium salt by weight, at least about 48.4% calcium salt by weight, at least about 50% calcium salt by weight, at least about 60% calcium salt by weight, at least about 70% calcium salt by weight, at least about 75% calcium salt by weight, at least about 80% calcium salt by weight, at least about 85% calcium salt by weight, at least about 90% calcium salt by weight, or at least about 95% calcium salt by weight.

Alternatively or in addition, such dry powder formulations may contain a calcium salt which provides Ca⁺² in an amount of at least about 3% Ca⁺² by weight, at least about 5% Ca⁺² by weight, at least about 7% Ca⁺² by weight, at least about 10% Ca⁺² by weight, at least about 11% Ca⁺² by weight, at least about 12% Ca⁺² by weight, at least about 13% Ca⁺² by weight, at least about 14% Ca⁺² by weight, at least about 15% Ca⁺² by weight, at least about 17% Ca⁺² by weight, at least about 20% Ca⁺² by weight, at least about 25% Ca⁺² by weight, at least about 30% Ca⁺² by weight, at least about 35% Ca⁺² by weight, at least about 40% Ca⁺² by weight, at least about 45% Ca⁺² by weight, at least about 50% Ca⁺² by weight, at least about 55% Ca⁺² by weight, at least about 60% Ca⁺² by weight, at least about 65% Ca⁺² by weight or at least about 70% Ca⁺² by weight.

When a dry powder salt formulation contains a calcium salt and a sodium salt the amount of sodium salt in the dry powder formulation can be dependent upon the desired calcium:sodium (mole:mole) ratio. For example, the dry powder formulation may contain at least about 1.6% sodium salt by weight, at least about 5% sodium salt by weight, at least about 10% sodium salt by weight, at least about 13% sodium salt by weight, at least about 15% sodium salt by weight, at least about 20% sodium salt by weight, at least about 24.4% sodium salt by weight, at least about 28% sodium salt by weight, at least about 30% sodium salt by weight, at least about 30.5% sodium salt by weight, at least about 35% sodium salt by weight, at least about 40% sodium salt by weight, at least about 45% sodium salt by weight, at least about 50% sodium salt by weight, at least about 55% sodium salt by weight, or at least about 60% sodium salt by weight.

Alternatively or in addition, dry powder salt formulations may contain a sodium salt which provides Na⁺ in an amount of at least about 0.1% Na⁺ by weight, at least about 0.5% Na⁺ by weight, at least about 1% Na⁺ by weight, at least about 2% Na⁺ by weight, at least about 3% Na⁺ by weight, at least about 4% Na⁺ by weight, at least about 5% Na⁺ by weight, at least about 6% Na⁺ by weight, at least about 7% Na⁺ by weight, at least about 8% Na⁺ by weight, at least about 9% Na⁺ by weight, at least about 10% Na⁺ by weight, at least about 11% Na⁺ by weight, at least about 12% Na⁺ by weight, at least about 14% Na⁺ by weight, at least about 16% Na⁺ by weight, at least about 18% Na⁺ by weight, at least about 20% Na⁺ by weight, at least about 22% Na⁺ by weight, at least about 25% Na⁺ by weight, at least about 27% Na⁺ by weight, at least about 29% Na⁺ by weight, at least about 32% Na⁺ by weight, at least about 35% Na⁺ by weight, at least about 40% Na⁺ by weight, at least about 45% Na⁺ by weight, at least about 50% Na⁺ by weight, or at least about 55% Na⁺ by weight.

Preferred excipients for dry powder salt formulations (such as mannitol, maltrodextrin or leucine) can be present in the formulations in an amount of about 50% or less (w/w). For example, a dry powder formulation may contain the amino acid leucine in an amount of about 50% or less by weight, about 45% or less by weight, about 40% or less by weight, about 35% or less by weight, about 30% or less by weight, about 25% or less by weight, about 20% or less by weight, about 18% or less by weight, about 16% or less by weight, about 15% or less by weight, about 14% or less by weight, about 13% or less by weight, about 12% or less by weight, about 11% or less by weight, about 10% or less by weight, about 9% or less by weight, about 8% or less by weight, about 7% or less by weight, about 6% or less by weight, about 5% or less by weight, about 4% or less by weight, about 3% or less by weight, about 2% or less by weight, or about 1% or less by weight. Exemplary excipients may include leucine, maltodextrin, mannitol, any combination of leucine, maltodextrin, and mannitol, or any other excipients disclosed herein or commonly used in the art.

In one embodiment, maltodextrin can be present in the dry powder salt formulations in an amount of about 50% or less (w/w). For example, a dry powder formulation may contain maltodextrin in an amount of about 50% or less by weight, about 45% or less by weight, about 40% or less by weight, about 35% or less by weight, about 30% or less by weight, about 25% or less by weight, about 20% or less by weight, about 18% or less by weight, about 16% or less by weight, about 15% or less by weight, about 14% or less by weight, about 13% or less by weight, about 12% or less by weight, about 11% or less by weight, about 10% or less by weight, about 9% or less by weight, about 8% or less by weight, about 7% or less by weight, about 6% or less by weight, about 5% or less by weight, about 4% or less by weight, about 3% or less by weight, about 2% or less by weight, or about 1% or less by weight.

In another embodiment, the dry powder may contain two different excipients (e.g., leucine, maltodextrin, mannitol, lactose, etc.). The excipients can be present in the formulation at a ratio of about 4:1, about 3:1, about 2:1, or about 1:1. Preferably, the dry powder formulation comprises leucine and maltodextrin as excipients in a 1:1 ratio.

For example, a liquid pharmaceutical formulation may contain from about 0.115 M to 1.15 M Ca²⁺ ion, from about 0.116 M to 1.15 M Ca²⁺ ion, from about 0.23 M to 1.15 M Ca²⁺ ion, from about 0.345 M to 1.15 M Ca²⁺ ion, from about 0.424 M to 1.15 M Ca²⁺ ion, from about 0.46 M to 1.15 M Ca²⁺ ion, from about 0.575 M to 1.15 M Ca²⁺ ion, from about 0.69 M to 1.15 M Ca²⁺ ion, from about 0.805 M to 1.15 M Ca²⁺ ion, from about 0.849 M to 1.15 M Ca²⁺ ion, or from about 1.035 M to 1.15 M Ca²⁺ ion. The solubility of certain calcium salts (e.g., calcium carbonate, calcium citrate) can limit the preparation of solutions. In such situations, the liquid formulation may be in the form of a suspension that contains the equivalent amount of calcium salt that would be needed to achieve the desired molar concentration.

When the salt formulation contains a sodium salt, such as a formulation that contains a calcium salt and a sodium salt, the Na⁺ ion in a liquid pharmaceutical formulation can be dependent upon the desired Ca²⁺:Na⁺ (mole:mole) ratio. For example, the liquid formulation may contain from about 0.053 M to 0.3 M Na⁺ ion, from about 0.075 M to 0.3 M Na⁺ ion, from about 0.106 M to 0.3 M Na⁺ ion, from about 0.15 M to 0.3 M Na⁺ ion, from about 0.225 M to 0.3 M Na⁺ ion, from about 0.008 M to 0.3 M Na⁺ ion, from about 0.015 M to 0.3 M Na⁺ ion, from about 0.016 M to 0.3 M Na⁺ ion, from about 0.03 M to 0.3 M Na⁺ ion, from about 0.04 M to 0.3 M Na⁺ ion, from about 0.08 M to 0.3 M Na⁺ ion, from about 0.01875 M to 0.3 M Na⁺ ion, from about 0.0375 M to 0.3 M Na⁺ ion, from about 0.075 M to 0.6 M Na⁺ ion, from about 0.015 M to 0.6 M Na⁺ ion, or from about 0.3 M to 0.6 M Na⁺ ion.

The compositions of some preferred calcium lactate or calcium citrate formulations are presented in Table 2. The compositions disclosed in Table 2 are non-limiting examples of calcium lactate or calcium citrate formulations of the invention.

TABLE 2
Liquid formulations

| | Tonicity (1X = | Ca-lactate | Ca-lactate | NaCl | NaCl |
|---|---|---|---|---|---|
| Formulation | isotonic) | (%) | (M) | (%) | (M) |
| 1 | 0.5X | 0.76 | 0.035 | 0.23 | 0.039 |
| 2 | 1X | 1.5 | 0.070 | 0.45 | 0.077 |
| 3 | 2X | 3.0 | 0.14 | 0.90 | 0.15 |
| 4 | 4X | 6.1 | 0.28 | 1.8 | 0.31 |
| 5 | 6X | 9.1 | 0.42 | 2.7 | 0.46 |
| 6 | 8X | 12 | 0.56 | 3.6 | 0.62 |
| 7 | 0.5X | 1.4 | 0.065 | 0.048 | 0.0082 |
| 8 | 1X | 2.9 | 0.13 | 0.10 | 0.016 |
| 9 | 2X | 5.7 | 0.26 | 0.19 | 0.033 |
| 10 | 3X | 9.0 | 0.41 | 0.30 | 0.052 |
| 11 | 4X | 11 | 0.52 | 0.38 | 0.065 |
| 12 | 8X | 23 | 1.0 | 0.77 | 0.13 |

Dry Powder formulations

| | Formulation composition | | | | | |
|---|---|---|---|---|---|---|
| Formulation | | Excipient | | Calcium | | Sodium |
| # | Excipient | (wt %) | Calcium salt | salt (wt %) | Sodium salt | salt (wt %) |
| 13 | Leucine | 50.0 | Calcium lactate | 37.0 | Sodium chloride | 13.0 |
| 14 | Leucine | 50.0 | Calcium chloride | 19.5 | Sodium citrate | 30.5 |
| 15 | Leucine | 10.0 | Calcium lactate | 66.6 | Sodium chloride | 23.4 |
| 16 | Leucine | 10.0 | Calcium chloride | 35.1 | Sodium citrate | 54.9 |
| 17 | Leucine | n.a. | Calcium lactate | 74.0 | Sodium chloride | 26.0 |
| 18 | Leucine | n.a. | Calcium chloride | 39.0 | Sodium citrate | 61.0 |
| 19 | Leucine | 10.0 | Calcium lactate | 58.6 | Sodium chloride | 31.4 |
| 20 | Maltodextrin | 10.0 | Calcium lactate | 58.6 | Sodium chloride | 31.4 |
| 21 | Mannitol | 10.0 | Calcium lactate | 58.6 | Sodium chloride | 31.4 |
| 22 | Lactose | 10.0 | Calcium lactate | 58.6 | Sodium chloride | 31.4 |
| 23 | Half leucine | 10.0 | Calcium lactate | 58.6 | Sodium chloride | 31.4 |
| | and half | | | | | |
| | maltodextrin | | | | | |
| | (wt basis) | | | | | |
| 24 | Half leucine | 20.0 | Calcium lactate | 52.1 | Sodium chloride | 27.9 |
| | and half | | | | | |
| | maltodextrin | | | | | |
| | (wt basis) | | | | | |
| 25 | Leucine | 20.0 | Calcium lactate | 52.1 | Sodium chloride | 27.9 |
| 26 | Leucine | 12.0 | Calcium lactate | 57.3 | Sodium chloride | 30.7 |
| 27 | Leucine | 8.0 | Calcium lactate | 59.9 | Sodium chloride | 32.1 |

### Methods

Treatment of Infectious Disease

The invention provides methods for treatment, prophylaxis and/or reducing contagion of infectious diseases of the respiratory tract (e.g., viral infections, bacterial infections). An effective amount of a calcium lactate or calcium citrate formulation is administered to the respiratory tract of an individual (e.g., a mammal, such as a human or other primate, or domesticated animal, such as pigs, cows, sheep, chickens). Preferably, the calcium lactate or calcium citrate formulation is administered by inhalation of an aerosol. The method can be used for treatment, prophylaxis and/or reducing contagion of bacterial or viral infections of the respiratory tract, for example, viral pneumonia, bacterial pneumonia, influenza, pharyngitis, bronchitis, laryngo-tracheo bronchitis, brochiolitis, and the like.

The invention provides methods for treatment (including prophylactic treatment) of pulmonary diseases, such as asthma (e.g., allergic/atopic, childhood, late-onset, cough-variant, or chronic obstructive), airway hyperresponsiveness, allergic rhinitis (seasonal or non-seasonal), brochiectasis, chronic bronchitis, emphysema, chronic obstructive pulmonary disease, cystic fibrosis, early life wheezing, and the like, and for the treatment (including prophylactic treatment) of acute exacerbations of these chronic diseases, such as exacerbations caused by a viral infection (e.g., influenza virus such as Influenza virus A or Influenza virus B, parainfluenza virus, respiratory syncytial virus, rhinovirus, adenovirus, metapneumovirus, coxsackie virus, echo virus, corona virus, herpes simplex virus, cytomegalovirus, and the like), bacterial infections (e.g., Streptococcus pneumoniae, which is commonly referred to as pneumococcus, Staphylococcus aureus, Streptococcus agalactiae, Streptococcus pyogenes, Haemophilus influenzae, Haemophilus parainfluenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Serratia marcescens, Burkholderia cepacia, Burkholderia pseudomallei, Bacillus anthracis, Bacillus cereus, Stenotrophomonas maltophilia, a bacterium from the citrobacter family, a bacterium from the ecinetobacter family, Mycobacterium tuberculosis, Bordetella pertussis, and the like), fungal infections (e.g., Histoplasma capsulatum, Cryptococcus neoformans, Pneumocystis jiroveci, Coccidioides immitis, Candida albicans, Pneumocystis jirovecii (which causes pneumocystis pneumonia (PCP), also called pneumocystosis), and the like), parasitic infections (e.g., Toxoplasma gondii, Strongyloides stercoralis, and the like), or environmental allergens and irritants (e.g., aeroallergens, including pollen, house-dust mites, animal dander such as cat dander, molds, cockroaches, airborne particulates, and the like).

In one aspect, the invention is a method for treating an individual with a bacterial infection of the respiratory tract or exhibiting symptoms of a bacterial infection of the respiratory tract, comprising administering to the respiratory tract of the individual an effective amount of a calcium lactate or calcium citrate formulation of the invention.

In some embodiments, the individual is infected by a bacteria selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii Burkholderia spp., methicillin-resistant Staphylococcus aureus, Stenotrophomonas maltophilia and combinations thereof, all of which can cause pneumonia. In particular embodiments, the individual is infected by Streptococcus pneumoniae, Klebsiella pneumoniae or Pseudomonas aeruginosa. In a more particular embodiment, the individual is infected by Streptococcus pneumoniae. In other embodiments the individual is infected by Bacillus anthracis or Mycobacterium tuberculosis.

In certain embodiments, the respiratory tract infection is a bacterial infection, such as bacterial pneumonia. In certain embodiments, the bacterial infection is caused by a bacterium selected from the group consisting of Streptococcus pneumoniae (also referred to as pneumococcus), Staphylococcus aureus, Streptococcus agalactiae, Streptococcus pyogenes, Haemophilus influenzae, Haemophilus parainfluenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Serratia marcescens, Burkholderia cepacia, Burkholderia pseudomallei, Bacillus anthracis, Bacillus cereus, Bordatella pertussis, Stenotrophomonas maltophilia, a bacterium from the citrobacter family, a bacterium from the ecinetobacter family, and Mycobacterium tuberculosis.

In certain embodiments, the respiratory tract infection is a viral infection, such as influenza or viral pneumonia. In certain embodiments, the viral infection is caused by a virus selected from the group consisting of influenza virus (e.g., Influenza virus A, Influenza virus B), respiratory syncytial virus, adenovirus, metapneumovirus, cytomegalovirus, parainfluenza virus (e.g., hPIV-1, hPIV-2, hPIV-3, hPIV-4), rhinovirus, adenovirus, coxsackie virus, echo virus, corona virus, herpes simplex virus, SARS-coronavirus, and smallpox.

In certain embodiments, the respiratory tract infection is a fungal infection. In certain embodiments, the fungal infection is caused by a fungus selected from the group consisting of Histoplasma capsulatum, Cryptococcus neoformans, Pneumocystis jiroveci, Coccidioides immitis, Candida albicans, and Pneumocystis jirovecii (which causes pneumocystis pneumonia (PCP), also called pneumocystosis).

In certain embodiments, the respiratory tract infection is a parasitic infection. In certain embodiments, the parasitic infection is caused by a parasite selected from the group consisting of Toxoplasma gondii and Strongyloides stercoralis.

In another aspect, the invention provides a method for treating (including prophylactically treating) an individual with a pulmonary disease (e.g., an individual having a pulmonary disease, exhibiting symptoms of a pulmonary disease, or susceptible to a pulmonary disease), comprising administering to the respiratory tract of the individual an effective amount of a pharmaceutical formulation comprising a calcium salt and a sodium salt, wherein the ratio of Ca⁺² to Na⁺ is from about 4:1 (mole:mole) to about 16:1 (mole:mole).

In another aspect, the invention provides a method for treating (including prophylactically treating) an acute exacerbation of a chronic pulmonary disease in an individual, comprising administering to the respiratory tract of the individual in need thereof (e.g., an individual having an acute exacerbation of a pulmonary disease, exhibiting symptoms of an acute exacerbation of a pulmonary disease, or susceptible to an acute exacerbation of a pulmonary disease) an effective amount of a pharmaceutical formulation comprising a calcium salt and a sodium salt, wherein the ratio of Ca⁺² to Na⁺ is from about 4:1 (mole:mole) to about 16:1 (mole:mole). Exemplary pulmonary diseases include asthma (e.g., allergic/atopic, childhood, late-onset, cough-variant, or chronic obstructive), airway hyperresponsiveness, allergic rhinitis (seasonal or non-seasonal), bronchiectasis, chronic bronchitis, emphysema, chronic obstructive pulmonary disease, cystic fibrosis, early life wheezing, and the like.

In certain embodiment, influenza is caused by either the influenza A or the influenza B virus.

In certain embodiments, an influenza-like illness is caused by RSV, rhinovirus, adenovirus, parainfluenza, human coronaviruses (including the virus that causes severe acute respiratory syndrome) and metapneumovirus.

In certain embodiments, ventilator associate pneumonia (VAP), ventilator associated tracheobronchitis (VAT), or hospital acquired pneumonia (HAP), is caused by pneumoniae, S. pneumoniae, S. aureus, non-typeable Haemophilus influenzae (NTHI), psuedominas aeruginosa, Acinetobacter spp., E coli, Candida spp (a fungus), Serratia, Enterobacter spp, and Stenotrophomonas. Alternatively, VAP or VAT can be caused by Gram-positive or Gram-negative bacteria associated with causing pneumonia.

In certain embodiments, community associated pneumonia (CAP) is caused by at least one of the following bacteria: Moraxella catarralis, Mycoplasma pneumoniae, Chlamydophilia pneumonia, or Chlamydia pneumoniae, strep pneumonia, Haemophilus influenzae, chlamydophia, mycoplasma, and Legionella. Alternatively, or in addition to the previously mentioned bacteria, CAP may also be caused by at least one of the following fungi: Coccidiomycosis, histoplasmosis, and cryptococcocus. Alternatively, CAP can be caused by Gram-positive or Gram-negative bacteria associated with causing pneumonia.

In certain embodiments, an acute exacerbation of a patient with asthma is caused by an upper respiratory tract viral infection or Gram-positive or Gram-negative bacteria associated with pneumonia, including CAP. Alternatively, or in addition, an acute exacerbation can be caused by allergens or environmental factors such as house dust mites, Ova, or pollen. An acute exacerbation of a patient with COPD is caused by the same causes as for asthma, and additionally by Haemophilus influenzae, pneumococcus, and moraxella. Mild exacerbations of CF can be caused by all of the above, in addition to the opportunitistic bacterial pathogens, such as Pseudomonas aeruginosa, Burkholderia cepacia, Burkholderia pseudomallei, and the like, that characterize CF airway colonization, and also by atypical mycobacteria and Stenotrophomonas.

In another aspect, the invention is a method for treating an individual with a viral infection of the respiratory tract or an individual exhibiting symptoms of a viral infection of the respiratory tract, comprising administering to the respiratory tract of the individual an effective amount of a calcium lactate or calcium citrate formulation of the invention. In some embodiments, the individual is infected by a virus selected from the group consisting of influenza virus, respiratory syncytial virus, adenovirus, metapneumovirus, cytomegalovirus, parainfluenza virus, rhinovirus, coronoaviruses (e.g., SARS-coronavirus), poxviruses (e.g., smallpox), and herpes simplex virus.

Preferably, the method of treating an infection of the respiratory tract comprises administering to an individual that has an infection of the respiratory tract or is exhibiting symptoms of a respiratory tract infection, an effective amount of a calcium lactate or calcium citrate formulation. More preferably, the calcium lactate or calcium citrate formulation also comprises a sodium salt, such as sodium chloride. Suitable calcium lactate and calcium citrate formulations, including formulations that contain calcium lactate and a sodium salt or calcium citrate and a sodium salt, are described herein.

Prophylaxis

In another aspect, the invention is a method for prophylaxis or prevention of an infection of the respiratory tract comprising administering to the respiratory tract of an individual at risk for infection of the respiratory tract by a pathogen (e.g., bacteria, virus) an effective amount of a calcium lactate or calcium citrate formulation. The method can be used to prevent or to decrease the rate or incidence of infection by a pathogen (e.g., bacteria, virus) that causes an infection of the respiratory tract.

The individual to be treated may be at risk for infection by a bacteria selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, Burkholderia spp., methicillin-resistant Staphylococcus aureus, Stenotrophomonas maltophilia and combinations thereof. In particular embodiments, the individual is at risk for infection by Streptococcus pneumoniae, Klebsiella pneumoniae or Pseudomonas aeruginosa. In a more particular embodiment, the individual is at risk for infection by Streptococcus pneumoniae.

In another aspect, the invention is a method for prophylaxis or prevention of infection by a virus selected from the group consisting of influenza virus, respiratory syncytial virus, adenovirus, metapneumovirus, cytomegalovirus, herpes simplex virus, coronaviruses (e.g., SARS-coronavirus), rhinovirus, parainfluenza, and poxviruses (e.g., smallpox).

Generally, individuals are at risk for infection by a pathogen (e.g., virus, bacteria) that causes infection of the respiratory tract when they are exposed to such a pathogen more frequently then the general population, or have a diminished capacity to resist infection. Individuals who are at risk for such an infection include, for example, health care workers, individuals who are immunosuppressed (e.g., medically, due to other infections, or for other reasons), patients in an intensive care unit, elderly and young (e.g., infants) individuals, individuals with chronic underlying respiratory disease (e.g., asthma, chronic bronchitis, chronic obstructive pulmonary disease, cystic fibrosis) individuals who have had surgery or traumatic injury, and care givers and family members of infected persons.

Preferably, the method of preventing an infection of the respiratory tract comprises administering to an individual at risk for an infection of the respiratory tract an effective amount of a calcium lactate or calcium citrate formulation. More preferably, the calcium lactate or calcium citrate formulation also comprises a sodium salt, such as sodium chloride. Suitable calcium lactate and calcium citrate formulations, including formulations that contain calcium lactate and a sodium salt or calcium citrate and a sodium salt, are described herein.

Reducing Contagion

The invention provides methods for reducing contagion (e.g., reducing transmission) of infection of the respiratory tract (e.g., viral infections, bacterial infections) comprising administering to the respiratory tract (e.g., lungs, nasal cavity) of an individual infected with a pathogen that causes infection of the respiratory tract, exhibiting symptoms of an infection of the respiratory tract, at risk for infection of the respiratory tract or at risk for infection by a pathogen (e.g., bacteria, virus) that causes infection of the respiratory tract, an effective amount of a calcium lactate or calcium citrate formulation.

In some embodiments, the individual may have an infection of the respiratory tract caused by a bacterial infection, exhibit symptoms of an infection of the respiratory tract or be at risk for such an infection as described herein. For example, the individual may be infected by or at risk for infection by a bacteria selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, Burkholderia spp., methicillin-resistant Staphylococcus aureus, Stenotrophomonas maltophilia and combinations thereof. In particular embodiments, the individual is infected by or at risk of infection by Streptococcus pneumoniae, Klebsiella pneumoniae or Pseudomonas aeruginosa. In a more particular embodiment, the individual is infected by or at risk for infection by Streptococcus pneumoniae.

In other embodiments, the individual may be infected by or at risk for infection by a virus selected from the group consisting of influenza virus, respiratory syncytial virus, adenovirus, metapneumovirus, cytomegalovirus, herpes simplex virus, coronaviruses (e.g., SARS-coronavirus), rhinovirus, parainfluenza, and poxviruses (e.g., smallpox).

Preferably, the method for reducing contagion of infection of the respiratory tract comprises administering to an individual that has an infection of the respiratory tract or is exhibiting symptoms of an infection of the respiratory tract, an effective amount of a calcium lactate or calcium citrate formulation. More preferably, the calcium lactate or calcium citrate formulation also comprises a sodium salt, such as sodium chloride. Suitable calcium lactate and calcium citrate formulations, including formulations that contain calcium lactate and a sodium salt or calcium citrate and a sodium salt, are described herein.

### Treatment of Chronic Disease

The invention provides methods for treatment of chronic respiratory and pulmonary diseases including asthma (e.g., allergic/atopic, childhood, late-onset, cough-variant, or chronic obstructive), airway hyperresponsiveness, allergic rhinitis (seasonal or non-seasonal), bronchiectasis, chronic bronchitis, emphysema, chronic obstructive pulmonary disease, cystic fibrosis, early life wheezing, and the like. An effective amount of a calcium lactate or calcium citrate formulation is administered to the respiratory tract of an individual (e.g., a mammal, such as a human or other primate, or domesticated animal, such as pigs, cows, sheep, chickens). Preferably, the calcium lactate or calcium citrate formulation is administered by inhalation of an aerosol. The method can be used for treatment of chronic respiratory and pulmonary diseases, for example cystic fibrosis.

Preferably, the method of treating a chronic pulmonary or respiratory disease comprises administering to an individual that has the chronic disease, an effective amount of a calcium lactate or calcium citrate formulation. More preferably, the calcium lactate or calcium citrate formulation also comprises a sodium salt, such as sodium chloride. Suitable calcium lactate and calcium citrate formulations, including formulations that contain calcium lactate and a sodium salt or calcium citrate and a sodium salt, are described herein.

### Prevention of Acute Exacerbations

The invention provides methods for preventing acute exacerbations of a chronic pulmonary disease in an individual, comprising administering to the respiratory tract of the individual in need thereof (e.g., an individual having an acute exacerbation of a pulmonary disease, exhibiting symptoms of an acute exacerbation of a pulmonary disease, or susceptible to an acute exacerbation of a pulmonary disease) an effective amount of a pharmaceutical formulation comprising calcium citrate or calcium lactate as active ingredients. Exemplary pulmonary diseases include asthma (e.g., allergic/atopic, childhood, late-onset, cough-variant, or chronic obstructive), airway hyperresponsiveness, allergic rhinitis (seasonal or non-seasonal), bronchiectasis, chronic bronchitis, emphysema, chronic obstructive pulmonary disease, cystic fibrosis, early life wheezing, and the like.

The invention also provides methods for preventing acute exacerbations of chronic pulmonary disease and preventing bronchoconstriction and bronchospasms due to antigen exposure (e.g., allergen, pathogen, and other environmental stimulants) in patients with chronic pulmonary disorders. An effective amount of a calcium lactate or calcium citrate formulation is administered to the respiratory tract of an individual (e.g., a mammal, such as a human or other primate, or domesticated animal, such as pigs, cows, sheep, chickens). Preferably, the calcium lactate or calcium citrate formulation is administered by inhalation of an aerosol. The method can be used for treatment of chronic respiratory and pulmonary diseases, for example cystic fibrosis.

Preferably, the methods of treating or preventing acute exacerbations due to aeroallergens comprises administering to an individual that has an enhanced propensity for bronchoconstriction and bronchospasms due to antigen exposure (e.g., allergen, pathogen, and other environmental stimulants), an effective amount of a calcium lactate or calcium citrate formulation. More preferably, the calcium lactate or calcium citrate formulation also comprises a sodium salt, such as sodium chloride. Suitable calcium lactate and calcium citrate formulations, including formulations that contain calcium lactate and a sodium salt or calcium citrate and a sodium salt, are described herein.

The calcium containing salts function to slow allergen and pathogen passage through the airway lining fluid (ALF) of the lungs by modulating the viscoelasticity of the ALF. The calcium containing salts facilitate the natural clearance of foreign matter by the mucocilliary escalator, and other clearance mechanisms in the pulmonary tract. By reducing the allergen and pathogen load in the epithelium, inflammation is reduced. Reduced inflammation leads to reduced production of toxic biproducts that are known to lead to bronchoconstriction and bronchospasms. Thus, administration of calcium containing salts reduce the frequency and/or severity of episodes of bronchoconstriction and bronchospasms in patients with chronic pulmonary disorders.

### Administering Calcium Lactate and Calcium Citrate Formulations

The calcium lactate formulations and calcium citrate formulations are intended for administration to the respiratory tract (e.g., to the mucosal surface of the respiratory tract), and can be administered in any suitable form, such as a solution, a suspension, a spray, a mist, a foam, a gel, a vapor, droplets, particles, or a dry powder form. Preferably the calcium lactate formulation and calcium citrate formulation is aerosolized for administration to the respiratory tract. Calcium lactate formulations and calcium citrate formulations can be aerosolized using any suitable method and/or device, and many suitable methods and devices are conventional and well-known in the art. For example, calcium lactate formulations and calcium citrate formulations can be aerosolized for administration via the oral airways using a metered dose inhaler (e.g., a pressurized metered dose inhalers (pMDI) including HFA propellant, or a non-HFA propellant) with or without a spacer or holding chamber, a nebulizer, an atomizer, a continuous sprayer, an oral spray or a dry powder inhaler (DPI). Salt formulations can be aerosolized for administration via the nasal airways using a nasal pump or sprayer, a metered dose inhaler (e.g., a pressurized metered dose inhaler (pMDI) including HFA propellant, or a non-HFA propellant) with or without a spacer or holding chamber, a nebulizer with or without a nasal adapter or prongs, an atomizer, a continuous sprayer, or a dry powder inhaler (DPI). Salt formulations can also be delivered to the nasal mucosal surface via, for example, nasal wash and to the oral mucosal surfaces via, for example, an oral wash. Salt formulations can be delivered to the mucosal surfaces of the sinuses via, for example, nebulizers with nasal adapters and nasal nebulizers with oscillating or pulsatile airflows.

The geometry of the airways is an important consideration when selecting a suitable method for producing and delivering aerosols of salt formulations to the lungs. The lungs are designed to entrap particles of foreign matter that are breathed in, such as dust. There are three basic mechanisms of deposition: impaction, sedimentation, and Brownian motion (J. M. Padfield. 1987. In: D. Ganderton & T. Jones eds. Drug Delivery to the Respiratory Tract, Ellis Harwood, Chicherster, U.K.). Impaction occurs when particles are unable to stay within the air stream, particularly at airway branches. Impacted particles are adsorbed onto the mucus layer covering bronchial walls and eventually cleared from the lungs by mucocilliary action. Impaction in the upper airways mostly occurs with particles over 5 μm in aerodynamic diameter. Smaller particles (those less than about 3 μm in aerodynamic diameter) tend to stay within the air stream and be advected deep into the lungs by sedimentation. Sedimentation often occurs in the lower respiratory system where airflow is slower. Very small particles (those less than about 0.6 μm) can deposit by Brownian motion.

For administration, a suitable method (e.g., nebulization, dry powder inhaler) is selected to produce aerosols with the appropriate particle size for preferential delivery to the desired region of the respiratory tract, such as the deep lung (generally particles between about 0.6 microns and 5 microns in diameter), the upper airway (generally particles of about 3 microns or larger diameter), or the deep lung and the upper airway.

An effective amount of calcium lactate or calcium citrate formulation is administered to an individual in need thereof, such as an individual who has an infection of the respiratory tract, who is exhibiting symptoms of an infection of the respiratory tract or who is at risk for infection of the respiratory tract. Individuals who are hospitalized, and particularly those who are ventilated, are at risk for infection by pathogens that cause infection of the respiratory tract. An effective amount is an amount that is sufficient to achieve the desired therapeutic or prophylactic effect, such as an amount sufficient to reduce symptoms of infection, to reduce time to recovery, to reduce pathogens in an individual, to inhibit pathogens passing through the lung mucus or airway lining fluid, to decrease the incidence or rate of infection with pathogens that cause infection of the respiratory tract, to increase mucociliary clearance (e.g., as measured by sinticraphy) (Groth et al, Thorax, 43(5):360-365 (1988)) and/or to decrease the shedding of exhaled particles containing pathogens that cause infection of the respiratory tract. Because the calcium lactate or calcium citrate formulations are administered to the respiratory tract, generally by inhalation, the dose that is administered is related to the composition of the calcium lactate or calcium citrate formulation (e.g., calcium salt concentration), the rate and efficiency of aerosolization (e.g., nebulization rate and efficiency), and the time of exposure (e.g., nebulization time). For example, substantially equivalent doses can be administered using a concentrated liquid calcium lactate or calcium citrate formulation and a short (e.g., 5 minutes) nebulization time, or using a dilute liquid salt formulation and a long (e.g., 30 minutes or more) nebulization time, or using a dry powder formulation and a dry powder inhaler. The clinician of ordinary skill can determine appropriate dosage based on these considerations and other factors, for example, the individual's age, sensitivity, tolerance and overall well-being. The salt formulations can be administered in a single dose or multiple doses as indicated.

As described herein, it is believed that the therapeutic and prophylactic effects of the salt formulations are the result of an increased amount of cation (the cation of the salt, such as Ca²⁺) in the respiratory tract (e.g., lung) following administration of a salt formulation. Accordingly, since the amount of cation provided can vary depending upon the particular salt selected, dosing can be based on the desired amount of cation to be delivered to the lung. For example, one mole of calcium chloride (CaCl₂) dissociates to provide one mole of Ca²⁺, but one mole of tricalcium phosphate (Ca₃(PO₄)₂) can provide three moles of Ca²⁺. Generally, an effective amount of a salt formulation will deliver a dose of about 0.001 mg Ca⁺²/kg body weight/dose to about 2 mg Ca⁺²/kg body weight/dose, about 0.002 mg Ca⁺²/kg body weight/dose to about 2 mg Ca⁺²/kg body weight/dose, about 0.005 mg Ca⁺²/kg body weight/dose to about 2 mg Ca⁺²/kg body weight/dose, about 0.01 mg Ca⁺²/kg body weight/dose to about 2 mg Ca⁺²/kg body weight/dose, about 0.01 mg Ca⁺²/kg body weight/dose to about 60 mg Ca⁺²/kg body weight/dose, about 0.01 mg Ca⁺²/kg body weight/dose to about 50 mg Ca⁺²/kg body weight/dose, about 0.01 mg Ca⁺²/kg body weight/dose to about 40 mg Ca⁺²/kg body weight/dose, about 0.01 mg Ca⁺²/kg body weight/dose to about 30 mg Ca⁺²/kg body weight/dose, about 0.01 mg Ca⁺²/kg body weight/dose to about 20 mg Ca⁺²/kg body weight/dose, about 0.01 mg Ca⁺²/kg body weight/dose to about 10 mg Ca⁺²/kg body weight/dose, about 0.01 mg Ca⁺²/kg body weight/dose to about 5 mg Ca⁺²/kg body weight/dose, about 0.01 mg Ca⁺²/kg body weight/dose to about 2 mg Ca⁺²/kg body weight/dose, about 0.02 mg Ca⁺²/kg body weight/dose to about 2 mg Ca⁺²/kg body weight/dose, about 0.03 mg Ca⁺²/kg body weight/dose to about 2 mg Ca⁺²/kg body weight/dose, about 0.04 mg Ca⁺²/kg body weight/dose to about 2 mg Ca⁺²/kg body weight/dose, about 0.05 mg Ca⁺²/kg body weight/dose to about 2 mg Ca⁺²/kg body weight/dose, about 0.1 mg Ca⁺²/kg body weight/dose to about 2 mg Ca⁺²/kg body weight/dose, about 0.1 mg Ca⁺²/kg body weight/dose to about 1 mg Ca⁺²/kg body weight/dose, about 0.1 mg Ca⁺²/kg body weight/dose to about 0.5 mg Ca⁺²/kg body weight/dose, about 0.2 mg Ca⁺²/kg body weight/dose to about 0.5 mg Ca⁺²/kg body weight/dose, about 0.18 mg Ca⁺²/kg body weight/dose, about 0.001 mg Ca⁺²/kg body weight/dose, about 0.005 mg Ca⁺²/kg body weight/dose, about 0.01 mg Ca⁺²/kg body weight/dose, about 0.02 mg Ca⁺²/kg body weight/dose, or about 0.5 mg Ca⁺²/kg body weight/dose. In some embodiments, a salt formulation that comprises a calcium salt (e.g., calcium chloride, calcium lactate, calcium citrate) is administered in an amount sufficient to deliver a dose of about 0.1 mg Ca²⁺/kg body weight/dose to about 2 mg Ca²⁺/kg body weight/dose, or about 0.1 mg Ca²⁺/kg body weight/dose to about 1 mg Ca²⁺/kg body weight/dose, or about 0.1 mg Ca²⁺/kg body weight/dose to about 0.5 mg Ca²⁺/kg body weight/dose, or about 0.18 mg Ca²⁺/kg body weight/dose.

In some embodiments the amount of calcium delivered to the respiratory tract (e.g., lungs, respiratory airway) is about 0.005 mg/kg body weight to about 60 mg/kg body weight/dose, or about 0.01 mg/kg body weight/dose to about 50 mg/kg body weight/dose, about 0.01 mg/kg body weight/dose to about 40 mg/kg body weight/dose, about 0.01 mg/kg body weight/dose to about 30 mg/kg body weight/dose, about 0.01 mg/kg body weight/dose to about 20 mg/kg body weight/dose, about 0.01 mg/kg body weight/dose to about 10 mg/kg body weight/dose, about 0.1 mg/kg body weight/dose to about 10 mg/kg body weight/dose, about 0.2 mg/kg body weight/dose to about 0.5 mg/kg body weight/dose or about 1 mg/kg body weight/dose to about 10 mg/kg body weight/dose, or about 0.01 mg/kg body weight/dose to about 1 mg/kg body weight/dose, or about 0.1 mg/kg body weight/dose to about 1 mg/kg body weight/dose, or about 0.1 mg Ca²⁺/kg body weight/dose to about 2 mg Ca²⁺/kg body weight/dose, or about 0.1 mg Ca²⁺/kg body weight/dose to about 0.5 mg Ca²⁺/kg body weight/dose, or about 0.18 mg Ca²⁺ kg body weight/dose.

In other embodiments the amount of calcium delivered to the upper respiratory tract (e.g., nasal cavity) is about 0.01 mg/kg body weight/dose to about 60 mg/kg body weight/dose, or about 0.01 mg/kg body weight/dose to about 50 mg/kg body weight/dose, about 0.01 mg/kg body weight/dose to about 40 mg/kg body weight/dose, about 0.01 mg/kg body weight/dose to about 30 mg/kg body weight/dose, about 0.01 mg/kg body weight/dose to about 20 mg/kg body weight/dose, 0.01 mg/kg body weight/dose to about 10 mg/kg body weight/dose, about 0.1 mg/kg body weight/dose to about 10 mg/kg body weight/dose, or about 1 mg/kg body weight/dose to about 10 mg/kg body weight/dose, or about 0.01 mg/kg body weight/dose to about 1 mg/kg body weight/dose, or about 0.1 mg/kg body weight/dose to about 1 mg/kg body weight/dose, about 0.001 mg Ca⁺²/kg body weight/dose to about 2 mg Ca⁺²/kg body weight/dose, about 0.002 mg Ca⁺²/kg body weight/dose to about 2 mg Ca⁺²/kg body weight/dose, about 0.005 mg Ca⁺²/kg body weight/dose to about 2 mg Ca⁺²/kg body weight/dose, about 0.01 mg Ca⁺²/kg body weight/dose to about 2 mg Ca⁺²/kg body weight/dose, about 0.01 mg Ca⁺²/kg body weight/dose to about 60 mg Ca⁺²/kg body weight/dose, about 0.01 mg Ca⁺²/kg body weight/dose to about 50 mg Ca⁺²/kg body weight/dose, about 0.01 mg Ca⁺²/kg body weight/dose to about 40 mg Ca⁺²/kg body weight/dose, about 0.01 mg Ca⁺²/kg body weight/dose to about 30 mg Ca⁺²/kg body weight/dose, about 0.01 mg Ca⁺²/kg body weight/dose to about 20 mg Ca⁺²/kg body weight/dose, about 0.01 mg Ca⁺²/kg body weight/dose to about 10 mg Ca⁺²/kg body weight/dose, about 0.01 mg Ca⁺²/kg body weight/dose to about 5 mg Ca⁺²/kg body weight/dose, about 0.01 mg Ca⁺²/kg body weight/dose to about 2 mg Ca⁺²/kg body weight/dose, about 0.02 mg Ca⁺²/kg body weight/dose to about 2 mg Ca⁺²/kg body weight/dose, about 0.03 mg Ca⁺²/kg body weight/dose to about 2 mg Ca⁺²/kg body weight/dose, about 0.04 mg Ca⁺²/kg body weight/dose to about 2 mg Ca⁺²/kg body weight/dose, about 0.05 mg Ca⁺²/kg body weight/dose to about 2 mg Ca⁺²/kg body weight/dose, about 0.1 mg Ca⁺²/kg body weight/dose to about 2 mg Ca⁺²/kg body weight/dose, about 0.1 mg Ca⁺²/kg body weight/dose to about 1 mg Ca⁺²/kg body weight/dose, about 0.1 mg Ca⁺²/kg body weight/dose to about 0.5 mg Ca⁺²/kg body weight/dose, about 0.2 mg Ca⁺²/kg body weight/dose to about 0.5 mg Ca⁺²/kg body weight/dose, about 0.18 mg Ca⁺²/kg body weight/dose, about 0.001 mg Ca⁺²/kg body weight/dose, about 0.005 mg Ca⁺²/kg body weight/dose, about 0.01 mg Ca⁺²/kg body weight/dose, about 0.02 mg Ca⁺²/kg body weight/dose, or about 0.5 mg Ca⁺²/kg body weight/dose

In some embodiments, a salt formulation that comprises a sodium salt (e.g., sodium chloride) is administered in an amount sufficient to deliver a dose of about 0.001 mg Na⁺/kg body weight/dose to about 10 mg Na⁺/kg body weight/dose, or about 0.01 mg Na⁺/kg body weight/dose to about 10 mg Na⁺/kg body weight/dose, or about 0.1 mg Na⁺/kg body weight/dose to about 10 mg Na⁺/kg body weight/dose, or about 1.0 mg Na⁺/kg body weight/dose to about 10 mg Na⁺/kg body weight/dose, or about 0.001 mg Na⁺/kg body weight/dose to about 1 mg Na⁺/kg body weight/dose, or about 0.01 mg Na⁺/kg body weight/dose to about 1 mg Na⁺/kg body weight/dose, about 0.1 mg Na⁺/kg body weight/dose to about 1 mg Na⁺/kg body weight/dose, about 0.2 mg Na⁺/kg body weight/dose to about 0.8 mg Na⁺/kg body weight/dose, about 0.3 mg Na⁺/kg body weight/dose to about 0.7 mg Na⁺/kg body weight/dose, or about 0.4 mg Na⁺/kg body weight/dose to about 0.6 mg Na⁺/kg body weight/dose.

In some embodiments the amount of sodium delivered to the respiratory tract (e.g., lungs, respiratory airway) is about 0.001 mg/kg body weight/dose to about 10 mg/kg body weight/dose, or about 0.01 mg/kg body weight/dose to about 10 mg/kg body weight/dose, or about 0.1 mg/kg body weight/dose to about 10 mg/kg body weight/dose, or about 1 mg/kg body weight/dose to about 10 mg/kg body weight/dose, or about 0.001 mg/kg body weight/dose to about 1 mg/kg body weight/dose, or about 0.01 mg/kg body weight/dose to about 1 mg/kg body weight/dose, or about 0.1 mg/kg body weight/dose to about 1 mg/kg body weight/dose.

In other embodiments the amount of sodium delivered to the upper respiratory tract (e.g., nasal cavity) is about 0.001 mg/kg body weight/dose to about 10 mg/kg body weight/dose, or about 0.01 mg/kg body weight/dose to about 10 mg/kg body weight/dose, or about 0.1 mg/kg body weight/dose to about 10 mg/kg body weight/dose, or about 1 mg/kg body weight/dose to about 10 mg/kg body weight/dose, or about 0.001 mg/kg body weight/dose to about 1 mg/kg body weight/dose, or about 0.01 mg/kg body weight/dose to about 1 mg/kg body weight/dose, or about 0.1 mg/kg body weight/dose to about 1 mg/kg body weight/dose.

Suitable intervals between doses that provide the desired therapeutic effect can be determined based on the severity of the condition (e.g., infection), overall well being of the subject and the subject's tolerance to the salt formulations and other considerations. Based on these and other considerations, a clinician can determine appropriate intervals between doses. Generally, a salt formulation is administered once, twice or three times a day, as needed.

If desired or indicated, a calcium lactate or calcium citrate formulation can be administered with one or more other therapeutic agents, such as any one or more of the mucoactive agents, surfactants, cough suppressants, expectorants, steroids, brochodilators, antihistamines, antibiotics, antiviral agents described herein. The other therapeutic agents can be administered by any suitable route, such as orally, parenterally (e.g., intravenous, intraarterial, intramuscular, or subcutaneous injection), topically, by inhalation (e.g., intrabronchial, intranasal or oral inhalation, intranasal drops), rectally, vaginally, and the like. The calcium lactate or calcium citrate formulation can be administered before, substantially concurrently with, or subsequent to administration of the other therapeutic agent. Preferably, the calcium lactate or calcium citrate formulation and the other therapeutic agent are administered so as to provide substantial overlap of their pharmacologic activities.

### EXEMPLIFICATION

### Example 1

### Calcium Citrate: Dry Powder

a. Formulation

The dry powder formulation comprised 50.0% leucine, 19.5% calcium chloride and 30.5% sodium citrate (weight percent (%)). This corresponds to a calcium to sodium molar ratio of 1 to 2 (Ca:Na, 1:2).

b. Process

i. Materials

Calcium chloride dihydrate and L-leucine were obtained from Sigma-Aldrich Co. (St. Louis, Mo.), and sodium citrate dihydrate from J. T. Baker (Phillipsburg, N.J.). Deionized (DI) water was from a Milli-Q water purification system (Millipore Corp., Billerica, Mass.). Liquid feeds were prepared with the soluble salts calcium chloride and sodium citrate as starting materials. Upon spray drying and thus liquid evaporation, the solution undergoes a precipitation reaction to produce calcium citrate and sodium chloride. The formulation contained 50.0% leucine, 19.5% calcium chloride and 30.5% sodium citrate (weight percent (%)). This was prepared by first dissolving 2.51 g of leucine in 1.0 L of DI water, then 1.74 g of sodium citrate dihydrate, and finally 1.30 g of calcium chloride dihydrate. The materials were completely dissolved in the water at room temperature, with continual stirring. A Niro Mobile Minor spray dryer (GEA Process Engineering Inc., Columbia, Md.) was used. Nitrogen was employed as the process drying gas. The gas inlet temperature was set to 140° C., with the outlet temperature reading about 72° C. The gas rate was 97 to 101 kg/hour and the liquid feed rate ranged from about 28 to about 30 ml/minute. The solutions were kept agitated throughout the process. Spray dried powders were collected in a vessel at the outlet of the cyclone, with the yield being about 62%.

c. In Vitro Characterization

i. Materials

A cell culture model of Influenza infection was used to study the effects of different nebulized solutions on viral infection. Calu-3 cells (American Type Culture Collection, Manassas, Va.) were cultured on permeable membranes (12 mm Transwells; 0.4 μm pore size, Corning, Lowell, Mass.) until confluent (membrane fully covered with cells) and air-liquid interface (ALI) cultures were established by removing the apical media and culturing at 37° C./5% CO₂. Cells were cultured for >2 weeks at ALI before each experiment. Prior to each experiment the apical surface of each Transwell was washed 3× with PBS.

For the delivery of formulations, capsules (QUALI-V-I, hypromellose, Size 2; Qualicaps, Europe S.A., Madrid, Spain) were filled and the weight of each capsule before and after exposure was recorded to determine the emitted dose for each capsule preparation. Capsules were punctured with a 2-prong puncture fork and immediately loaded into a dry powder inhaler fitted to dry powder sedimentation chamber. Dry powder was pulled into the sedimentation chamber from capsules using an automated vacuum system in which the vacuum was turned on for 0.3 second in three sequential intervals spaced 1 minute apart. Infections and washes were performed as described above for liquid formulations.

Immediately after exposure to formulations, the basolateral media (media on the bottom side of the Transwell) was replaced with fresh media. Triplicate wells were exposed to each formulation in each test. A second cell culture plate was exposed to the same formulations to quantify the delivery of total salt or calcium to cells. One hour after exposure, cells were infected with 10 μL of Influenza A/WSN/33/1 at a multiplicity of infection of 0.1-0.01 (0.1-0.01 virions per cell). Four hours after aerosol treatment, the apical surfaces were washed to remove excess formulation and unattached virus and cells were cultured for an additional 20 hours at 37° C. plus 5% CO₂. The next day (24 h after aerosol treatment) virus released onto the apical surface of infected cells was collected in culture media or PBS and the concentration of virus in the apical wash was quantified by TCID₅₀ (50% Tissue Culture Infectious Dose) assay. The TCID₅₀ assay is a standard endpoint dilution assay that is used to quantify how much of a given virus is present in a sample.

ii. Calcium Citrate Dry Powder Inhibited Influenza Infection

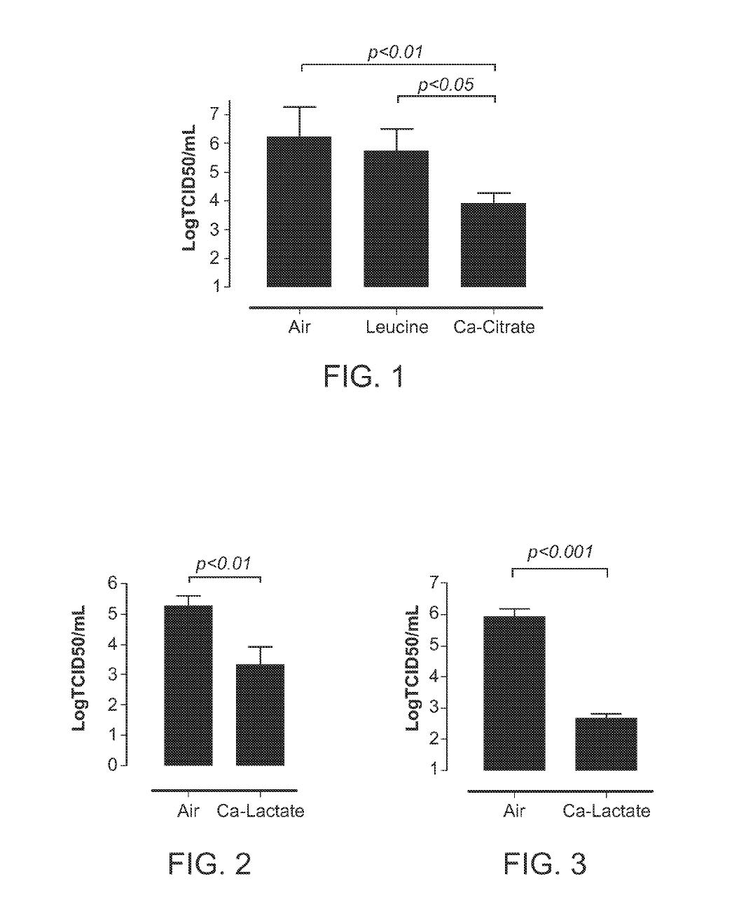

Calu-3 cells were exposed to Ca-citrate dry powder formulation or a dry powder consisting of 100% Leucine. Cells exposed to the Ca-citrate dry powder had reduced Influenza titers in the apical washes 24 hours after dosing compared to both untreated control cells (p<0.01) and cells exposed to control leucine dry powder [p<0.05; (FIG. 1; One-way ANOVA and Tukey's Multiple Comparison Test)]. Thus, dry powder formulations containing Ca-citrate can be effectively used to limit Influenza infection in vitro.

### Example 2

### Calcium Lactate: Liquid

a. Formulation

The liquid formulation contained 3.0% (w/v) calcium lactate (or 0.14M calcium lactate) and 0.90% (w/v) sodium chloride (or 0.15M sodium chloride). This corresponds to a calcium to sodium molar ratio of 1.0 to 1.1 (Ca:Na, 1:1.1).

b. Solution Preparation

i. Materials

Calcium lactate pentahydrate was obtained from Spectrum Chemicals (Gardena, Calif.) and sodium chloride from Sigma-Aldrich Co. (St. Louis, Mo.). Deionized (DI) water was from a Milli-Q water purification system (Millipore Corp., Billerica, Mass.).

ii. Liquid Formulation Preparation

The calcium lactate liquid formulation was prepared with stock solutions of calcium lactate and sodium chloride. A 0.14M [3.0% (wt/vol)] solution of calcium lactate in 0.15M NaCl [0.90% (w/v) NaCl] was formulated by dissolving 0.853 g of calcium lactate pentahydrate in 20 mL of 0.15M NaCl. The NaCl solution was made first by diluting 3 mL of a 1M NaCl stock solution in 17 mL of sterile water. Solutions were agitated until all solids were dissolved and stored at room temperature.

c. In Vitro Characterization

i. Materials

A cell culture model of Influenza infection was used to study the effects of different nebulized solutions on viral infection. Calu-3 cells (American Type Culture Collection, Manassas, Va.) were cultured on permeable membranes (12 mm Transwells; 0.4 μm pore size, Corning, Lowell, Mass.) until confluent (membrane is fully covered with cells) and air-liquid interface (ALI) cultures were established by removing the apical media and culturing at 37° C./5% CO₂. Cells were cultured for >2 weeks at ALI before each experiment. Prior to each experiment the apical surface of each Transwell was washed 3× with PBS. Cells were subsequently exposed to nebulized formulations with an in-house developed Sedimentation chamber and Series 8900 nebulizers (Slater Labs, Arvin, Calif.). Immediately after exposure, the basolateral media (media on the bottom side of the Transwell) was replaced with fresh media. Triplicate wells were exposed to each formulation in each test. A second cell culture plate was exposed to the same formulations to quantify the delivery of total salt or calcium to cells. One hour after exposure, cells were infected with 10 μL of Influenza A/WSN/33/1 at a multiplicity of infection of 0.1-0.01 (0.1-0.01 virions per cell). Four hours after aerosol treatment, the apical surfaces were washed to remove excess formulation and unattached virus and cells were cultured for an additional 20 h at 37° C. plus 5% CO₂. The next day (24 hours after aerosol treatment) virus released onto the apical surface of infected cells was collected in culture media or PBS and the concentration of virus in the apical wash was quantified by TCID₅₀ (50% Tissue Culture Infectious Dose) assay. The TCID₅₀ assay is a standard endpoint dilution assay that is used to quantify how much of a given virus is present in a sample.

ii. Calcium Lactate Liquid Formulation Inhibited Influenza Infection

Calu-3 cells were exposed to a liquid formulation of Ca-lactate (0.14M) in isotonic saline (0.15M) and infected with Influenza A/WSN/33/1. The viral titer on the apical surface of cells was determined 24 hours after dosing. The Ca-lactate formulations significantly reduced viral infection compared to the untreated control (FIG. 2; p<0.01 compared to untreated (Air) control; unpaired t-test test) indicating that Ca-lactate salts can effectively inhibit influenza infection.

3. Calcium Lactate: Dry powder
a. Formulation

A dry powder formulation comprised of 50% leucine, 37% calcium lactate and 13% sodium chloride (weight %) was prepared. This corresponds to a calcium to sodium molar ratio of 1.0 to 1.3 (Ca:Na, 1.0:1.3).

b. Process

i. Materials

Calcium lactate pentahydrate was obtained from Spectrum Chemicals (Gardena, Calif.), while L-leucine and sodium chloride were purchased from Sigma-Aldrich Co. (St. Louis, Mo.). Deionized (DI) water was from a Milli-Q water purification system (Millipore Corp., Billerica, Mass.). Liquid feeds were prepared with the soluble salts calcium lactate and sodium chloride. The formulation contained 50% leucine, 37% calcium lactate and 13% sodium chloride (weight %). This was prepared by first dissolving 2.51 g of leucine in 1.0 L of DI water, then 0.65 g of sodium chloride, and finally 2.62 g of calcium lactate pentahydrate. The materials were completely dissolved in the water at room temperature, with continual stirring. A Niro Mobile Minor spray dryer (GEA Process Engineering Inc., Columbia, Md.) was used. Nitrogen was employed as the process drying gas. The gas inlet temperature was set to 140° C., with the outlet temperature reading about 75° C. The gas rate was 97 to 101 kg/hour and the liquid feed rate ranged from about 29 to about 32 ml/minute. The solutions were kept agitated throughout the process. Spray dried powders were collected in a vessel at the outlet of the cyclone, with the yield being about 65%.

c. In Vitro Characterization

The activity of the dry powder formulation was assessed using the Calu-3 cell infection assay.

For the delivery of formulations, capsules (QUALI-V-I, hypromellose, Size 2; Qualicaps, Europe S.A., Madrid, Spain) were filled and the weight of each capsule before and after exposure was recorded to determine the emitted dose for each capsule preparation. Capsules were punctured with a 2-prong puncture fork and immediately loaded into a dry powder inhaler fitted to an in-house developed dry powder sedimentation chamber. Dry powder was pulled into the sedimentation chamber from capsules using an automated vacuum system in which the vacuum was turned on for 0.3 seconds in three sequential intervals spaced 1 minute apart. Infections and washes were performed as described above for liquid formulations.

Immediately after exposure to formulations, the basolateral media (media on the bottom side of the Transwell) was replaced with fresh media. Triplicate wells were exposed to each formulation in each test. A second cell culture plate was exposed to the same formulations to quantify the delivery of total salt or calcium to cells. One hour after exposure, cells were infected with 10 μL of Influenza A/WSN/33/1 at a multiplicity of infection of 0.1-0.01 (0.1-0.01 virions per cell). Four hours after aerosol treatment, the apical surfaces were washed to remove excess formulation and unattached virus and cells were cultured for an additional 20 h at 37° C. plus 5% CO₂. The next day (24 h after aerosol treatment) virus released onto the apical surface of infected cells was collected in culture media or PBS and the concentration of virus in the apical wash was quantified by TCID₅₀ (50% Tissue Culture Infectious Dose) assay. The TCID₅₀ assay is a standard endpoint dilution assay that is used to quantify how much of a given virus is present in a sample.

ii. Ca-Lactate Dry Powder Formulation Inhibited Influenza Infection

Treatment with Ca-lactate dry powder reduced Influenza infection as shown by reduced viral titers in the apical washes 24 hours after dosing (FIG. 3; p<0.001 unpaired t-test). Coupled with data generated with liquid formulations, the data shows that Ca-lactate acts to reduce Influenza infection in both liquid and dry powder form.

### Example 4

### Bacteria Pass-Through Assay

### Method

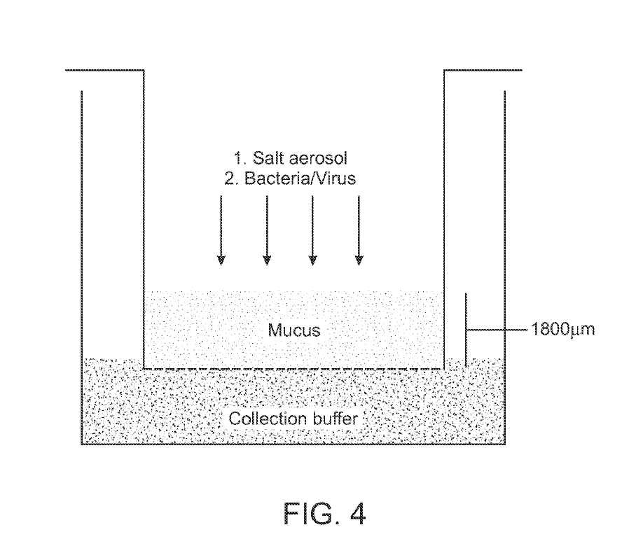

A pass-through model was used to test the effect of aerosolized dry powder formulations on bacterial movement across a mucus mimetic. This assay is a model for bacterial infection of the respiratory tract, because bacteria must cross the airway mucus to establish infection. In this model, 200 μL of 4% sodium alginate (Sigma-Aldrich, St. Louis, Mo.) was added to the apical surface of a 12 mm Costar Transwell membrane (Corning, Lowell, Mass.; 3.0 μm pore size) and subsequently exposed to dry powder formulations. Dry powders were aerosolized into the chamber using a dry powder insufflator (Penn-Century, Inc., Philadelphia, Pa.) and allowed to settle by gravity over a 5 minute period. Following this exposure, 10 μL of Klebsiella pneumoniae (˜10⁷ CFU/mL in saline) was added to the apical surface of the mimetic. At various time points after the addition of bacteria, aliquots of the basolateral buffer were removed and the number of bacteria in each aliquot was determined by serially diluting and plating on blood agar plates. A schematic of this method is shown in FIG. 4. The concentration of salt that was delivered to each Transwell was quantified by HPLC. For this purpose, empty wells of the 12 well cell culture plate that were next to each Transwell and were exposed to the same dose of formulation were rinsed with sterile water and diluted 1:1 with acetic acid to solubilize each dry powder.

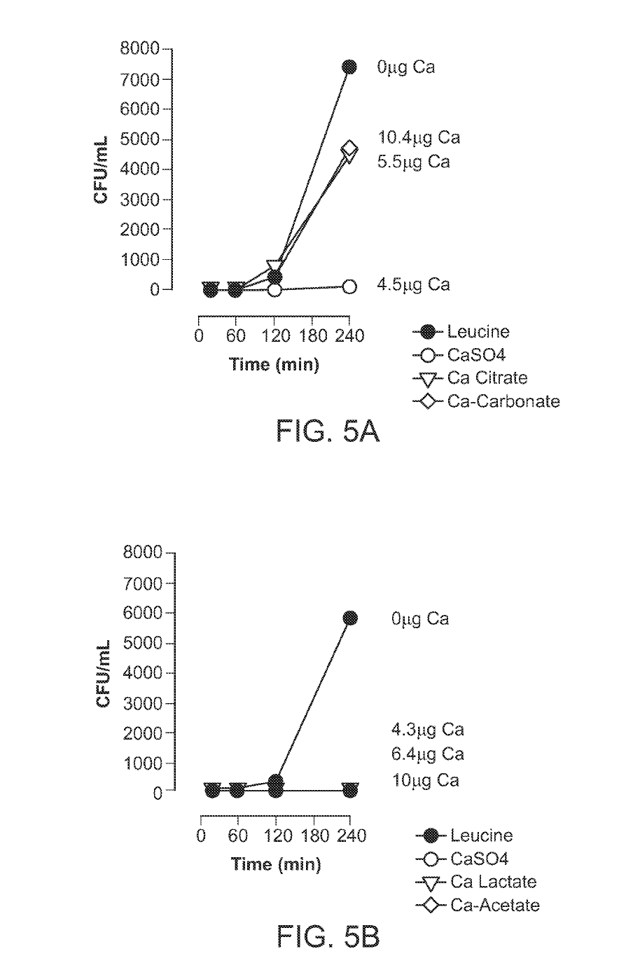

Dry powder formulations comprised of calcium salts with different solubility profiles, together with leucine and sodium chloride were screened for activity in the bacterial pass through model. The following dry powders were tested (wt %): 50% leucine/22% calcium chloride/28% sodium sulfate; 50% leucine/25.5% calcium chloride/24.5% sodium carbonate; 50% leucine/19.5% calcium chloride/30.5% sodium citrate; 50% leucine/37% calcium lactate/13% sodium chloride; and 50% leucine/33.75% calcium acetate/16.25% sodium chloride. The results for this study are shown in FIGS. 5A and 5B. The dry powders containing sulfate, acetate and lactate salts reduced the movement of bacteria across the mimetic (FIG. 5B), whereas the citrate and carbonate dry powders were less effective (FIG. 5A). These finding correlated with the known solubility of the calcium salts in water, suggesting that the failure of carbonate and citrate salts to inhibit the movement of K. pneumoniae is related to the solubility of these dry powders at the surface of the sodium alginate mimetic. The solubility of these salts from least soluble to most soluble is: calcium carbonate<calcium citrate<calcium sulfate<calcium lactate<calcium acetate.

### Example 6

### Dry Powder Formulations for Reducing Influenza Infection

Calu-3 cells were cultured on permeable membranes (12 mm Transwells; 0.4 μm pore size, Corning Lowell, Mass.) until confluent and air-liquid interface (ALI) cultures were established by removing the apical media and culturing at 37° C./5% CO₂. Cells were cultured for >2 weeks at ALI before each experiment. Prior to each experiment the apical surface of each Transwell was washed 3× with 500 μL/well PBS and the basolateral media (media on the bottom side of the Transwell) was replaced with 1.5 mL/well fresh media after dry powder exposure. Dry powder formulations were exposed to cells using an in-house developed Sedimentation chamber. Triplicate wells were exposed to each formulation in each test. At 1 hour post-exposure to dry powder formulation, cells were infected apically with 10 μL/well of Influenza A/WSN/33/1 at a multiplicity of infection of 0.1-0.01. Four hours after exposure, the apical surfaces were washed with 500 μL/well PBS to remove excess formulation and unattached virus and cells were cultured for an additional 20 h at 37° C. plus 5% CO₂. The next day (24 hours after infection) virus released onto the apical surface of infected cells was collected and the concentration of virus in the apical wash was quantified by TCID₅₀ (50% Tissue Culture Infectious Dose) assay.

After capsule exposure with the DPI Sedimentation Chamber, the final weight of each capsule was recorded to determine percent yield. The weight of new Qualicap capsules was recorded for each experiment. For each dry powder condition tested, two of these capsules were filled to appropriate fill weight. One of these capsules was used for cell exposure and the other was used for quantification. The dry powder conditions tested were 15 mg fill weight of the 100% Leucine and low, medium, high (5 mg, 15 mg, 60 mg, respectively) fill weights of the calcium lactate dry powder.

A: Ca-Lactate Reduces Influenza Infection in a Dose Responsive Manner.

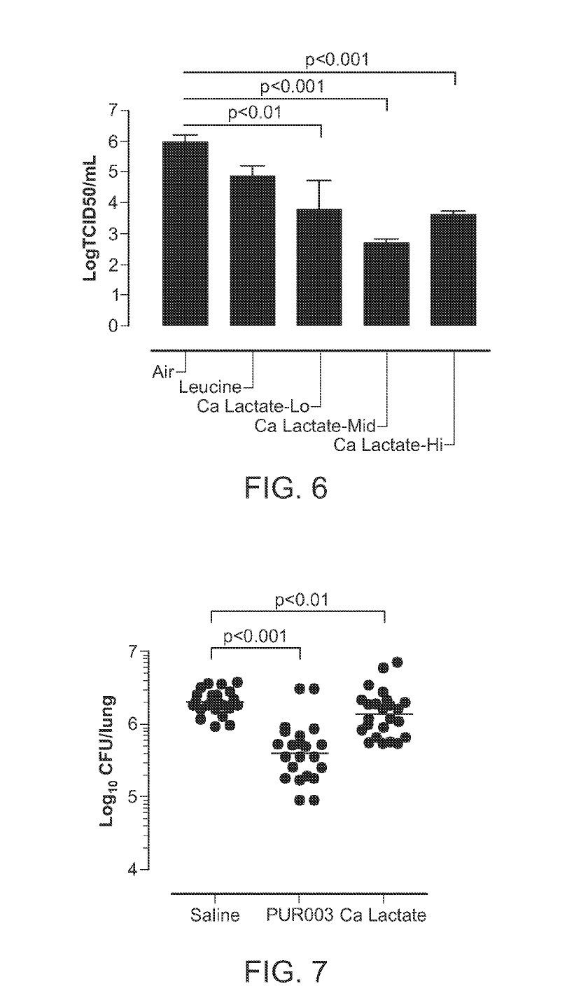

Calu-3 cells were exposed to a dose response of Ca-lactate (50% leucine, 37% calcium lactate and 13% sodium chloride) dry powders or a leucine control dry powder (100% leucine) to test whether calcium dry powders exhibited comparable efficacy to liquid formulations. 24 hours after treatment, the titer of virus in the apical washes from Calu-3 cells was determined by TCID₅₀ assay. As shown in FIG. 6, each of the Ca-lactate dry powder concentrations reduced the viral titer compared to the air control in a dose responsive manner (p<0.001 determined from one way ANOVA and Tukey multiple comparison post test).

### Example 7

### Calcium Lactate Formulations Effectively Reduce Bacterial Burden

Bacteria were prepared by growing cultures on tryptic soy agar (TSA) blood plates overnight at 37° C. plus 5% CO₂. Single colonies were resuspended to an OD₆₀₀˜0.3 in sterile PBS and subsequently diluted 1:4 in sterile PBS [˜2×10⁷ Colony forming units (CFU)/mL]. Mice were infected with 50 μL of bacterial suspension (˜1×10⁶ CFU) by intratracheal instillation while under anesthesia.

C57BL6 mice were exposed to aerosolized liquid formulations in a whole-body exposure system using either a high output prototype Pulmatrix nebulizer or Pari LC Sprint nebulizer connected to a pie chamber cage that individually holds up to 11 animals. Treatments were performed 2 hours before infection with Serotype 3 Streptococcus pneumoniae. Unless otherwise stated, exposure times were 3 minutes in duration. Twenty-four hours after infection mice were euthanized by pentobarbital injection and lungs were collected and homogenized in sterile PBS. Lung homogenate samples were serially diluted in sterile PBS and plated on TSA blood agar plates. CFU were enumerated the following day.

Mice were treated with isotonic saline or PUR003 (0.116M CaCl₂ in 0.15M NaCl; 1:1.3 Ca:Na ratio) two hours before infection with S. pneumoniae. Compared to control animals, PUR003 treated animals (approximate dose 1.9 mg/kg CaCl₂) exhibited 5-fold lower bacterial titers 24 hours after infection (FIG. 7), indicating a therapeutic benefit to the treatment. To determine if this effect was specific to formulations containing calcium chloride, we tested a similar formulation comprising calcium lactate (0.116M) dissolved in isotonic saline.

### Example 8

### Viral Replication Assay

This example demonstrates the efficacy of dry powder formulations comprising calcium salt, calcium lactate, calcium sulfate or calcium citrate dry powders with respect to treatment of influenza, parainfluenza or rhinovirus.

The PUR111, PUR112, and PUR113 powders were produced by spray drying utilizing a Mobile Minor spray dryer (Niro, GEA Process Engineering Inc., Columbia, Md.). All solutions had a solids concentration of 10 g/L and were prepared with the components listed in Table 3. Leucine and calcium salt were dissolved in DI water, and leucine and sodium salt were separately dissolved in DI water with the two solutions maintained in separate vessels. Atomization of the liquid feed was performed using a co-current two-fluid nozzle (Niro, GEA Process Engineering Inc., Columbia, Md.). The liquid feed was fed using gear pumps (Cole-Parmer Instrument Company, Vernon Hills, Ill.) into a static mixer (Charles Ross & Son Company, Hauppauge, N.Y.) immediately before introduction into the two-fluid nozzle. Nitrogen was used as the drying gas and dry compressed air as the atomization gas feed to the two-fluid nozzle. The process gas inlet temperature was 282° C. and outlet temperature was 98° C. with a liquid feedstock rate of 70 mL/min. The gas supplying the two-fluid atomizer was approximately 14.5 kg/hr. The pressure inside the drying chamber was at −2 “WC. Spray dried product was collected in a container from a filter device.

TABLE 3
Formulations used to evaluate efficacy

| | | | Ca:Na | |
|---|---|---|---|---|
| | | | molar | Manu- |
| Lot # | Formulation | Composition | ratio | facturing |
| 26-190-F | PUR111 | 10.0% leucine, 35.1% | 1:2 | Niro |
| | | calcium chloride, 54.9% | | |
| | | sodium citrate (Active | | |
| | | with 12.7% calcium ion) | | |
| 65-003-F | PUR112 | 10.0% leucine, 39.6% | 1:2 | Niro |
| | | calcium chloride, 50.4% | | |
| | | sodium sulfate (Active | | |
| | | with 14.3% calcium ion) | | |
| 65-009-F | PUR113 | 10.0% leucine, 58.6% | 1:2 | Niro |
| | | calcium lactate, 31.4% | | |
| | | sodium chloride (Active | | |
| | | with 10.8% calcium ion) | | |

A cell culture model of Influenza A/Panama/2007/99, human parainfluenza type 3 (hPIV3) or Rhinovirus (Rv16) infection was used to evaluate the efficacy of dry powder formulations. This model utilizes Calu-3 cells grown at air-liquid interface as a model of influenza infection of airway epithelial cells. Calu-3 cells were exposed to dry powders using a dry powder sedimentation chamber. The amount of calcium ion (Ca²⁺) delivered to each well was determined by HPLC using dry powder recovered from an empty well in the cell culture plate. The concentration of calcium deposited in each study is shown in Table 4.

TABLE 4
Calcium Deposition

| | PUR111 (μg Ca/cm²) | | | PUR112(μg Ca/cm²) | | | PUR113(μg Ca/cm²) | | |
|---|---|---|---|---|---|---|---|---|---|
| | Low | Medium | High | Low | Medium | High | Low | Medium | High |
| Influenza | 12.74 | 17.12 | 28.85 | 11.37 | 15.84 | 27.73 | 10.93 | 16.01 | 26.61 |
| Parainfluenza | 10.58 | 16.19 | 25.04 | 12.26 | 15.71 | 25.32 | 11.03 | 16.81 | 26.33 |
| Rhinovirus | 11.63 | 16.25 | 24.11 | 10.86 | 15.01 | 23.89 | 11.49 | 15.22 | 24.69 |

One hour after exposure, cells were infected with 10 μL of Influenza A/Panama/99/2007 at a multiplicity of infection of 0.1-0.01 (0.1-0.01 virions per cell), human parainfluenza type 3 (hPIV3) at a multiplicity of infection of 0.1-0.01 (0.1-0.01 virions per cell), or 10 μL of rhinovirus (Rv16) at a multiplicity of infection of 0.1-0.01 (0.1-0.01 virions per cell). Four hours after dry powder treatment, the apical surfaces were washed to remove excess formulation and unattached virus, and cells were cultured for an additional 20 hours at 37° C. plus 5% CO₂. The next day (24 hours after infection) virus released onto the apical surface of infected cells was collected in culture media and the concentration of virus in the apical wash was quantified by TCID₅₀ (50% Tissue Culture Infectious Dose) assay. The TCID₅₀ assay is a standard endpoint dilution assay that is used to quantify how much of a given virus is present in a sample. For each of the three dry powders, Calu-3 cells were exposed to three different Ca²⁺ doses and the replication of each virus was assessed.

Influenza

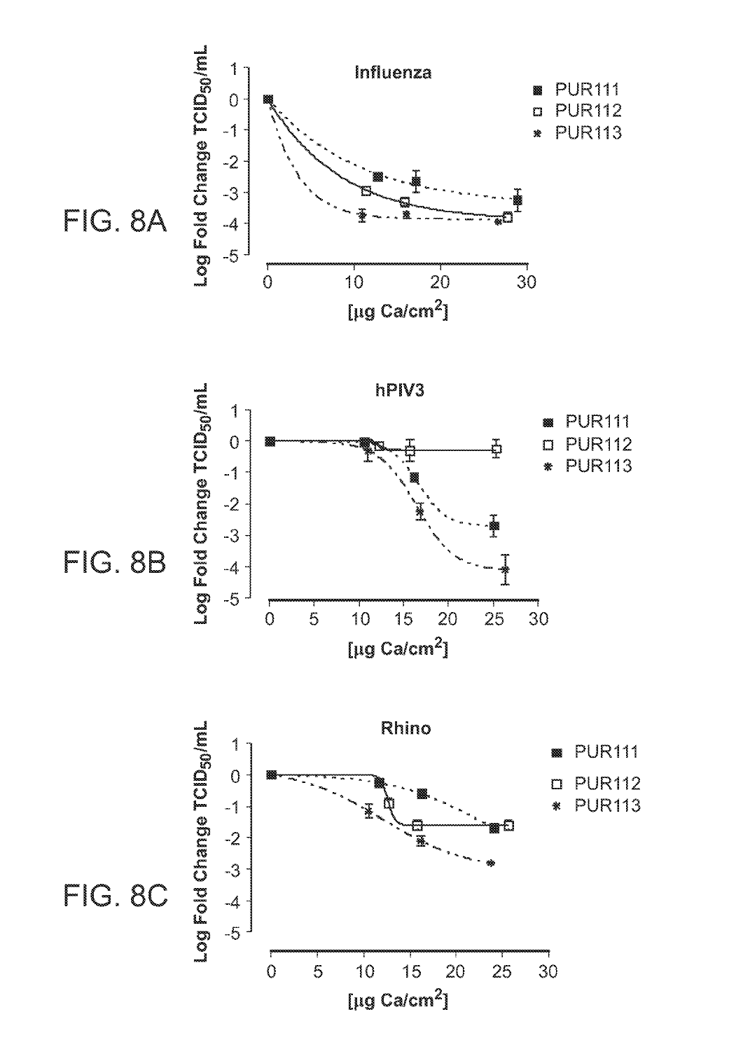

In the influenza model, all three dry powders significantly reduce viral titer to comparable levels at the highest dose tested: PUR111, PUR112, and PUR113 reduced viral titer up to 3.25, 3.80, and 3.95 log 10 TCID₅₀/mL, respectively (FIG. 8A). It is important to note that while at the highest dose tested these dry powders exhibited similar activity against influenza, at lower doses the data suggests the most efficacious dry powder was PUR113 (comprised of leucine, calcium lactate and sodium chloride). PUR113 reduced viral titers 3.70 and 3.75 log 10 TCID₅₀/mL at low and medium doses, whereas low doses of PUR111 and PUR112 reduced viral titer 2.50 and 2.95 log 10 TCID₅₀/mL, and mid doses of PUR111 and PUR112 reduced viral titers 2.65 and 3.30 log 10 TCID₅₀/mL, respectively.

Parainfluenza

PUR111, PUR112, and PUR113 were tested over a similar dose range against parainfluenza. The parainfluenza titer in the PUR112 treated cell cultures was comparable to the control cells (FIG. 8B) at doses of calcium similar to those used in the influenza experiment, indicating that the calcium sulfate based formulation may exhibit activity only against specific pathogens. In contrast, PUR111 and PUR113 treatment resulted in a dose dependent reduction in parainfluenza infection. At high doses, PUR111 and PUR113 reduced infection by 2.70 and 4.10 log 10 TCID₅₀/mL, respectively, compared to the control cells. Similarly, PUR113 exhibited greater efficacy than PUR111 at the middle dose tested, however, neither formulation reduced infection at the lowest dose tested (FIG. 8B; Table 4). Collectively, these data demonstrate that calcium based dry powder formulations effectively reduce the infectivity of parainfluenza. These effects are specific to certain calcium salts and the efficacious dose ranges differ significantly from that observed for influenza.

Rhinovirus

Influenza and parainfluenza are enveloped viruses. To test the broad spectrum activity of calcium dry powder formulations and extend these findings to nonenveloped viruses, the same dry powders were tested against rhinovirus. All three formulations reduced rhinovirus to some extent, with the PUR113 dry powder demonstrating the greatest activity. PUR113 treatment resulted in a significant, 2.80 log 10 TCID₅₀/mL viral reduction at the highest dose tested. Low and medium doses of this dry powder reduced titer 1.15 and 2.10 log 10 TCID₅₀/mL, respectively, compared to control cells. PUR111 and PUR112 treatment also reduced rhinovirus infection, albiet to a lesser extent than PUR113. At the highest dose tested, PUR111 reduced infection by 1.70 log 10 TCID50/mL and PUR112 reduced infection 1.60 log 10 TCID50/mL. Together these results indicate that calcium based dry powder formulations can be broadly applied to diverse viral infections.

The above data suggests that by increasing the delivered dose of calcium dry powder formulations exhibit more activity than was previously observed at lower doses. Influenza infection was reduced by all three dry powders tested, although the calcium lactate based formulation (PUR113) exhibited greater potentcy than the calcium sulfate (PUR112) and calcium citrate (PUR112) formulations. Additionally, across all three viral strains, PUR113 treatment resulted in the greatest reduction in viral titer. At higher doses PUR111 effectively reduced viral titer in all three viral strains, but the effect was much more pronounced with influenza and parainfluenza, suggesting a difference in mechanism that may be related to viral strain specificity. PUR112 treatment was active against parainfluenza, but exhibited better activity against both influenza and rhinovirus, suggesting that the specific calcium counterions may have some role in the optimal activity of the formulation.

The entire teachings of all documents cited herein are hereby incorporated herein by reference.

What is claimed is:

1. A pharmaceutical composition comprising as an active ingredient a calcium salt selected from the group consisting of calcium lactate and calcium citrate, wherein the pharmaceutical composition is suitable for inhalation.

2. The pharmaceutical composition of claim 1, further comprising a sodium salt.

3. (canceled)

4. The pharmaceutical composition of claim 2 wherein the ratio of calcium to sodium is between about 2:1 (mole:mole) to about 16:1 (mole:mole).

5-7. (canceled)

8. The pharmaceutical composition of claim 2 wherein the ratio of calcium to sodium is about 1:1 (mole:mole).

9. The pharmaceutical composition of claim 2 wherein the ratio of calcium to sodium is about 1:1.3 (mole:mole).

10. The pharmaceutical composition of claim 2 wherein the ratio of calcium to sodium is about 1:2 (mole:mole).

11-12. (canceled)

13. The pharmaceutical composition of claim 1, wherein the pharmaceutical composition is formulated to deliver a calcium salt dose of about 0.005 mg/kg body weight/dose to about 10 mg/kg body weight/dose to the lungs or nasal cavity.

14. The pharmaceutical composition of claim 1, wherein the pharmaceutical composition is formulated to deliver a calcium salt dose of about 0.1 mg/kg body weight/dose to about 2 mg/kg body weight/dose to the lungs or nasal cavity.

15. The pharmaceutical composition of claim 1 any, wherein the pharmaceutical composition is formulated to provide a sodium dose of about 0.001 mg/kg body weight/dose to about 10 mg/kg body weight/dose to the lungs or nasal cavity.

16. (canceled)

17. The pharmaceutical composition of claim 1, wherein the composition is a liquid formulation.

18. (canceled)

19. The pharmaceutical composition of claim 1, wherein the pharmaceutical composition is a dry powder.

20-21. (canceled)

22. The pharmaceutical composition of claim 19, wherein the calcium salt is present in a concentration of from about 20% to about 99 (w/w).

23. The pharmaceutical composition of claim 1 further comprising an additional therapeutic agent.

24. The pharmaceutical composition of claim 1 further comprising an excipient.

25. The pharmaceutical composition of claim 24, wherein said excipient is selected from the group consisting lactose, glycine, alanine, leucine, isolucine, trehalose, dipalmitoylphosphosphatidylcholine (DPPC), diphosphatidyl glycerol (DPPG), 1,2-Dipalmitoyl-sn-glycero-3-phospho-L-serine (DPPS), 1,2-Dipalmitoyl-sn-glycero-3-phosphocholine (DSPC), 1,2-Distearoyl-sn-glycero-3-phosphoethanolamine (DSPE), 1-palmitoyl-2-oleoylphosphatidylcholine (POPC), polyoxyethylene-9-lauryl ether, sorbitan trioleate (Span 85), glycocholate, surfactin, tyloxapol, sodium phosphate, dextran, dextrin, mannitol, maltodextrin, human serum albumin, recombinant human serum albumin, and biodegradable polymers.

26. The pharmaceutical composition of claim 1 wherein the composition is a unit dose composition.

27. A method for treating an infection of the respiratory tract, comprising administering to an individual having an infection of the respiratory tract, or exhibiting symptoms of an infection of the respiratory tract, an effective amount of a pharmaceutical composition of claim 1.

28. A method for prophylaxis of an infection of the respiratory tract, comprising administering to an individual at risk of contracting an infection of the respiratory tract an effective amount of a pharmaceutical composition of claim 1.

29. A method for reducing the spread of an infection of the respiratory tract, comprising administering to an individual having an infection of the respiratory tract, exhibiting symptoms of an infection of the respiratory tract or at risk of contracting an infection of the respiratory tract an effective amount of a pharmaceutical composition of claim 1.

30. A method for reducing the shedding of particles, comprising administering to an individual having an infection of the respiratory tract, exhibiting symptoms of an infection of the respiratory tract or at risk of contracting an infection of the respiratory tract an effective amount of a pharmaceutical composition of claim 1.

31-32. (canceled)
